SEQUENCE LISTING (9 sequences)

SEQ ID NO: 1 - protein, 16 aa, Artificial Sequence
  Synthetic
RFGAYLIQAGRMTPEG

SEQ ID NO: 2 - DNA, 18 bp, Artificial Sequence
  Synthetic
caccaccgccgagaccgc

SEQ ID NO: 3 - DNA, 18 bp, Artificial Sequence
  Synthetic
gctggccgggcttacctg

SEQ ID NO: 4 - DNA, 25 bp, Artificial Sequence
  Synthetic
ccttccttcttgggtaagttgtagc

SEQ ID NO: 5 - DNA, 29 bp, Artificial Sequence
  Synthetic
ccatacctaagagaagagtgacagaaatc

SEQ ID NO: 6 - DNA, 19 bp, Artificial Sequence
  Synthetic
cccaagcaatacccaaaga

SEQ ID NO: 7 - DNA, 21 bp, Artificial Sequence
  Synthetic
ccactttgctcttgacttcta

SEQ ID NO: 8 - DNA, 18 bp, Artificial Sequence
  Synthetic
tcacaagcagagcacaag

SEQ ID NO: 9 - DNA, 19 bp, Artificial Sequence
  Synthetic
gaaacagtccagcccatac

(19) United States
(12) Patent Application Publication
(10) Pub. No.: US 2021/0095254 A1
(43) Pub. Date: Apr. 1, 2021

(54) HIGHLY FUNCTIONAL MANUFACTURED STEM CELLS

(71) Applicants: Children's Medical Center Corporation, Boston, MA (US); TICEBA GmbH, Heidelberg (DE)

(72) Inventors: Markus H. Frank, Cambridge, MA (US); Andreas Kluth, Heidelberg (DE); Christoph Ganss, Heidelberg (DE)

(73) Assignees: Children's Medical Center Corporation, Boston, MA (US); TICEBA GmbH, Heidelberg (DE)

(21) Appl. No.: 16/835,144

(22) Filed: Mar. 30, 2020

Related U.S. Application Data
(63) Continuation-in-part of application No. PCT/US2020/025288, filed on Mar. 27, 2020.
(60) Provisional application No. 62/826,931, filed on Mar. 29, 2019.
(60) Provisional application No. 62/825,785, filed on Mar. 28, 2019.
(60) Provisional application No. 62/826,931, filed on Mar. 29, 2019.
(60) Provisional application No. 62/825,785, filed on Mar. 28, 2019.

(51) Int. Cl. C12N 5/0775; A61K 35/28; A61P 17/02; A61P 29/00; C12N 5/071

(52) U.S. Cl. CPC C12N 5/0668; A61K 35/28; C12N 5/0625; A61P 29/00; A61P 17/02

(57) ABSTRACT

Populations of synthetic ABCB5+ stem cells, wherein greater than 96.8% of the population is an in vitro progeny of physiologically occurring skin-derived ABCB5-positive mesenchymal stem cells are provided. Also provided are methods of making the synthetic cells and methods of use thereof.

### RELATED APPLICATIONS

This application claims the benefit under 35 U.S.C § 119(e) of U.S. Provisional Application Ser. No. 62/825,785, filed Mar. 28, 2019, entitled “HIGHLY FUNCTIONAL MANUFACTURED STEM CELLS” and of U.S. Provisional Application Ser. No. 62/826,931, filed Mar. 29, 2019, entitled “HIGHLY FUNCTIONAL MANUFACTURED STEM CELLS”, and this application is a continuation in part of international application number PCT/US2020/025288 filed Mar. 27, 2020, the entire contents of each of which are incorporated herein by reference.

### REFERENCE TO SEQUENCE LISTING

The instant application contains a Sequence Listing which has been submitted in ASCII format via EFS-Web and is hereby incorporated by reference in its entirety. The ASCII file, created on Aug. 21, 2020, is named C087570075US02-SEQ-HCL.txt and is 3 kilobytes in size.

### BACKGROUND OF INVENTION

Although poorly defined, self-renewing adult pluripotent mesenchymal stem cells (MSCs) reside within nearly all adult connective tissues, including the dermis [1, 2]. Their most important function is to maintain their niche environment, a critical requirement to protect their own stemness and long-term self-renewal capacity essential for tissue homeostasis, repair and organ maintenance [3].

The ATP-binding cassette sub-family B member 5, short ABCB5, also known as P-glycoprotein ABCB5 is a plasma membrane-spanning protein (Allikmets, et al., 1996). The ABC superfamily of active transporters, including transporters like ABCB1 (MDR1), ABCB4 (MDR2/3) and ABCG2 (Bcrp1, MXR1) which have been suggested to be responsible for causing drug resistance in cancer patients (Moitra and Dean, 2011), serves normal cellular transport, differentiation and survival functions in nonmalignant cell types. These well-known ABC transporters have been shown to be expressed at high levels on stem and progenitor cell populations. The efflux capacity for the fluorescent dyes Rhodamine123 and Hoechst 33342 mediated by these and related ABC transporters has been utilized for the isolation of such cell subsets from multiple tissues.

Recently, it was shown that ATP-binding cassette, sub-family B, member 5 (ABCB5) identifies a novel dermal immunomodulatory subpopulation, which in addition expresses MSC markers and exerts suppressive effects on effector T cells, while enhancing regulatory T-cells in vitro and in vivo [5]. ABCB5 belongs to the multiple drug resistant cell membrane anchored proteins also expressed on limbal stem cells of the eye where its absence results in blindness [6].

ABCB5 was confirmed to be a novel P-glycoprotein of the ABC transporter superfamily by additional structure analysis (Frank, et al., 2003). The designated ABCB5 protein located on chromosome 7p21-15.3 marks CD133-expressing progenitor cells among human epidermal melanocytes. The ABCB5 gene contains 19 exons and spans 108 kb of genomic DNA. The deduced 812-amino acid ABCB5 protein has 5 transmembrane helices flanked by both extracellular and intracellular ATP-binding domains.

Several characteristics are associated with the P-glycoprotein ABCB5 like the regulation of membrane potential and cell fusion of skin progenitor cells, the function as a rhodamine-123 efflux transporter and the marking of polyploid progenitor cell fusion hybrids, which contribute to culture growth and differentiation in human skin. In physiological skin progenitor cells, ABCB5 confers membrane hyperpolarization, and regulates as a determinant of membrane potential the propensity of this cell subpopulation to remain undifferentiated or to undergo differentiation (Frank, et al., 2005, Frank, et al., 2003). In addition, ABCB5-positive cells were shown to have anti-inflammatory, pro-angiogeneic and immunomodulatory properties (Schatton, et al., 2015, Webber, et al., 2017).

### SUMMARY OF THE INVENTION

It is shown here that the ABCB5⁺ stem cell populations can reliably be isolated from tissue and processed according to GMP standards to generate highly functional synthetic stem cells.

In some aspects a composition, comprising a population of synthetic ABCB5+ stem cells, wherein greater than 96% of the population is an in vitro progeny of physiologically occurring skin-derived ABCB5-positive mesenchymal stem cells is provided. In some embodiments greater than 96.5%, 97%, 97.5%, 98%, 98.5%, 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells. In some embodiments, 100% of the population is an in vitro progeny of physiologically occurring skin-derived ABCB5-positive mesenchymal stem cells.

In some embodiments greater than 90% of the synthetic stem cells in the population co-express CD90. In other embodiments the population of synthetic stem cells are capable of VEGF secretion under hypoxia as measured by ELISA. In other embodiments the population of synthetic stem cells are capable of IL-1RA secretion after co-culture with Mi-polarized macrophages. In other embodiments the population of synthetic stem cells induce decreased TNF-alpha and IL-12/IL-23p40 secretion, and increased IL-10 secretion, in macrophage co-culture relative to isolated physiologically occurring skin-derived ABCB5-positive mesenchymal stem cells. In other embodiments the population of synthetic stem cells possess multipotent differentiation capacity. In other embodiments the population of synthetic stem cells possess the capacity to differentiate into cells derived from all three germ layers, endoderm, mesoderm and ectoderm. In other embodiments the population of synthetic stem cells possess corneal epithelial differentiation capacity. In other embodiments the population of synthetic stem cells exhibit increased expression of stem cell markers including SOX2, NANOG and SOX3 relative to isolated physiologically occurring skin-derived ABCB5-positive mesenchymal stem cells. In other embodiments the population of synthetic stem cells exhibit decreased expression of mesenchymal stromal differentiation markers including MCAM, CRIG1 and ATXN1 relative to isolated physiologically occurring skin-derived ABCB5-positive mesenchymal stem cells. In other embodiments at least 5% of the population of synthetic stem cells includes an exogenous gene. In other embodiments at least 10%, 15%, 20%, 25%, 30%, 35%, 40%, 45%, 50%, 55%, 60%, 65%, 70%, 75%, 80%, 85%, 90%, or 95% of the population of synthetic stem cells includes an exogenous gene. In other embodiments the exogenous gene is a gene encoding a protein selected from the group consisting of tissue-specific homing factors, secreted tissue remodeling proteins, growth factors, cytokines, hormones and neurotransmitters. In other embodiments at least 5% of the population of synthetic stem cells comprise a modification in a gene. In other embodiments at least 10%, 15%, 20%, 25%, 30%, 35%, 40%, 45%, 50%, 55%, 60%, 65%, 70%, 75%, 80%, 85%, 90%, or 95% of the population of synthetic stem cells comprise a modification in a gene. In other embodiments the synthetic stem cells are modified by delivering a complex comprising a CRISPR RNA-guided nuclease and a gRNA that targets the gene. In yet other embodiments he modified gene is a gene selected from the group consisting of COL7A or defective genes in ABCB5+ cells.

The invention in some aspects is method for preparing a population of cells, by isolating a primary cells from skin tissue from a human subject; culturing the primary cells in culture medium until the cells produce enough progeny to reach greater than 60% confluence of mixed cells, harvesting the mixed cells, culturing the harvested mixed cells, reharvesting and culturing the cells through at least 5 passages until the population of cells reaches at least 99% manufactured synthetic cells and less than 10% is primary physiologically occurring skin-derived cells; and isolation of ABCB5-positive cells using an ABCB5+ antibody.

In some embodiments the method involves reharvesting and culturing the cells through at least 6, 7, 8, 9, 10, 11, 12, 13, 14, 15, or 16 passages. In other embodiments the method involves reharvesting and culturing the cells until the population of cells reaches at least 99.99% manufactured synthetic cells and less than 0.01% is primary physiologically occurring skin-derived cells. In other embodiments the method involves reharvesting and culturing the cells until the population of cells reaches at least 99.9995% manufactured synthetic cells and less than 0.0005% is primary physiologically occurring skin-derived cells. In other embodiments the method involves reharvesting and culturing the cells until the population of cells reaches at least 99.999997% manufactured synthetic cells and less than 0.000003% is primary physiologically occurring skin-derived cells. In other embodiments the isolation step involves ABCB5 antibody conjugated to magnetic beads. In other embodiments the cells are cultured in culture medium prepared with Ham's F-10 as basal medium. In other embodiments the cell confluence and cell morphology are evaluated at each cell expansion step. In other embodiments at least 3 days separates the final culture and isolation steps. In other embodiments the cells are harvested using EDTA.

In some aspects a method for inducing tissue generation is provide. The method involves promoting differentiation of an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells into a differentiated tissue.

In other aspects the invention is a method for promoting syngeneic transplants comprising administering to a subject having a syngeneic transplant an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells.

In other aspects the invention is a method for treating peripheral arterial occlusive disease (PAOD), comprising administering to a subject having PAOD an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells in an effective amount to treat the disease.

In other aspects the invention is a method for treating acute-on-chronic liver failure (AOCLF), comprising administering to a subject having AOCLF an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells in an effective amount to treat the disease.

In other aspects the invention is a method for treating limbal stem cell deficiency (LSCD), comprising administering to a subject having LSCD an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells in an effective amount to treat the disease.

In other aspects the invention is a method for treating corneal disease, comprising administering to a subject having corneal disease an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells in an effective amount to treat the disease.

In other aspects the invention is a method for treating epidermolysis bullosa (EB), comprising administering to a subject having EB an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells in an effective amount to treat the disease.

In other aspects the invention is a method for cutaneous wound healing, comprising contacting a wound with an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells in an effective amount to promote healing of the wound. In some embodiments the isolated population of synthetic ABCB5+ stem cells are seeded onto a matrix or scaffold. In other embodiments the matrix is a polymeric mesh or sponge, a polymeric hydrogel, or a collagen matrix.

In other aspects the invention is a method comprising administering to a subject having an organ transplant an effective amount of isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells to promote allograft survival.

In other aspects the invention is a method of treating autoimmune disease, comprising administering to a subject having autoimmune disease an effective amount of isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells to treat the autoimmune disease.

In other aspects the invention is a method of treating liver disease, comprising administering to a subject having a liver disease an effective amount of an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells to treat the liver disease.

In other aspects the invention is a method of treating a neurodegenerative disease, comprising administering to a subject having a neurodegenerative disease an effective amount of an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells to treat the neurodegenerative disease and wherein the neurodegenerative disease is associated with an immune response against host cells.

In other aspects the invention is a method of treating cardiovascular disease, comprising administering to a subject having cardiovascular disease an effective amount of an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells to treat the cardiovascular disease and, wherein the cardiovascular disease is associated with tissue remodeling.

In other aspects the invention is a method of treating kidney disease, comprising administering to a subject having a kidney disease an effective amount of an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells to treat the kidney disease.

In other aspects the invention is a method of treating an inflammatory disorder, comprising administering to a subject having an inflammatory disorder, an effective amount of an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells to treat the inflammatory disorder. In some embodiments the inflammatory disorder is selected from the group consisting of cardiovascular disease, ischemic stroke, Alzheimer disease and aging.

In other aspects the invention is a method of treating a musculoskeletal disorder, comprising administering to a subject having an inflammatory disorder, an effective amount of an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells to treat the musculoskeletal disorders. In some embodiments the musculoskeletal disorder is a genetic muscular dystrophy. In other embodiments the population of synthetic stem cells is the synthetic cells described herein.

In other aspects the invention is a method for cellular reprogramming, by using the population of synthetic stem cells as claimed in any one of claims 1-18 as a substrate for cellular reprogramming by pluripotency.

In other aspects the invention is a population of synthetic stem cells as described herein and further comprising an exogenous PAX6 gene.

Use of a population of stem cells of the invention for treating any of the disorders as described herein, tissue engineering, or wound healing is also provided as an aspect of the invention.

A method for manufacturing a medicament of a population of stem cells of the invention for treating any of the disorders as described herein, tissue engineering, or wound healing is also provided.

In other aspects a method of treating a hyper-inflammatory disorder, by administering to a subject having a hyper-inflammatory disorder, an effective amount of an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells to treat the hyper-inflammatory disorder. In some embodiments the hyper-inflammatory disorder is a disorder associated with a virally induced cytokine storm. In some embodiments the subject has a SARS infection. In some embodiments the hyper-inflammatory disorder is a disorder associated with sepsis, systemic inflammatory response syndrome (SIRS), cachexia, septic shock syndrome, traumatic brain injury (e.g., cerebral cytokine storm), graft versus host disease (GVHD), or the result of treatment with activated immune cells, e.g., IL-2 activated T cells, T cells activated with anti-CD19 Chimeric Antigen Receptor (CAR) T cells.

In other aspects the invention is a method of treating a SARS infection in a subject, by administering to the subject, an effective amount of an isolated population of synthetic ABCB5+ stem cells to treat the SARS infection in the subject. In some embodiments the SARS infection is a SARS-CoV-2 infection. In some embodiments greater than 99%, 99.5%, 99.7%, 99.9%, 99.99%, 99.998%, 99.999%, or 99.999997% of the population of synthetic ABCB5+ stem cells is an in vitro progeny of physiologically occurring skin-derived ABCB5-positive mesenchymal stem cells. In some embodiments the cells are administered intravenously.

In other aspects, the invention is a method of treating a subject having an infectious disease, by identifying a subject having an infectious disease and at risk of or having a cytokine storm associated with an infectious disease; and administering an isolated population of synthetic ABCB5+ stem cells to treat the subject. In some embodiments the infectious disease is caused by a coronavirus. In some embodiments the coronavirus is SARS-CoV-2. In some embodiments greater than 99%, 99.5%, 99.7%, 99.9%, 99.99%, 99.998%, 99.999%, or 99.999997% of the population of synthetic ABCB5+ stem cells is an in vitro progeny of physiologically occurring skin-derived ABCB5-positive mesenchymal stem cells. In some embodiments the cells are administered intravenously.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1: Flow chart summarizing the manufacturing process of synthetic stem cells.

FIGS. 2A-2G: ABCB5+ MSCs may belong to upper rather than lower fibroblast lineage. (FIG. 2A) Heatmap depicting transcriptome profiling of samples (n=3) from low (2-3) and high (above 10) passaged ABCB5+-derived MSCs. The color reflects the log 2 scale of relative expression. (FIG. 2B) Heatmap depicting genes involved in the maintenance of stemness from early and late passaged ABCB5+-derived MSCs. (FIG. 2C) A clear co-localization of ABCB5 with the stem cell marker SSEA-4 was observed in a distinct subpopulation of dermal cells. (FIGS. 2D-2E) Microphotographs of human skin subjected to double immunofluorescence staining for ABCB5 and the two marker proteins of “upper lineage” fibroblasts revealed a co-expression of ABCB5 with DPP4 (CD26) and a partial co-localization of ABCB5 and PRDM1 (BLIMP1). (FIG. 2F) A co-localization of ABCB5 with the stem cell marker POU5F1 (OCT-4). (FIG. 2G) ABCB5 was consistently not found co-expressed with the lower lineage fibroblast and myofibroblasts marker α-smooth muscle actin (α-SMA). Nuclei of all studied skin sections were counterstained with DAPI. Scale bars: 50 μm; e=epidermis; d=dermis. Dashed line delineates epidermal from dermal layers.

### DESCRIPTION OF THE INVENTION

In some aspects the invention is a population of in vitro manufactured skin-derived ABCB5-positive mesenchymal stem cells. These cells represent a significant advancement over isolated primary cell populations of skin-derived ABCB5-positive mesenchymal stem cells. Typically once primary cells are isolated and cultured in vitro, the cells lose important properties associated with the original primary cells. It has been discovered, according to the invention, that, under appropriate conditions, ABCB5+ stem cells isolated from human tissue can be passaged in culture to produce populations of cells that are structurally and functionally distinct from the original primary cells isolated from the tissue. These cells are referred to herein as synthetic or manufactured ABCB5+ stem cells. These cells are in vitro manufactured such that nearly all cells are in vitro progeny of physiologically occurring skin-derived ABCB5-positive mesenchymal stem cells that never existed in the context of the human body. Rather, they are newly created according to newly established culture methods. Although these cell populations are distinct from the original primary cells they are highly functional pluripotent cells, which have many therapeutic uses.

The synthetic ABCB5+ stem cells, as used herein, have one or more of the following properties:
co-express CD90>90%;
are capable of VEGF secretion under hypoxia as measured by ELISA;
are capable of IL-1RA secretion after co-culture with Mi-polarized macrophages;
induce decreased TNF-alpha and IL-12/IL-23p40 secretion, and increased IL-10 secretion, in macrophage co-culture;
possess multipotent differentiation capacity; or
different gene expression profile.

The compositions of the invention are populations of cells. The term “population of cells” as used herein refers to a composition comprising at least two, e.g., two or more, e.g., more than one, synthetic ABCB5+ stem cells, and does not denote any level of purity or the presence or absence of other cell types, unless otherwise specified. In an exemplary embodiment, the population is substantially free of other cell types. In another exemplary embodiment, the population comprises at least two cells of the specified cell type, or having the specified function or property, for example as listed above.

In some embodiments, the synthetic stem cells induce decreased TNF-alpha and IL-12/IL-23p40 secretion. These properties of the cells are important for their anti-inflammatory functions. As a result of these cytokines, the cells are useful for treating a number of inflammatory disease. In other embodiments the cells produce increased IL-10 secretion, in macrophage co-culture. The production of IL-10 is important for supporting the tolerogenic functions of the synthetic stem cells.

The cells of the invention also possess multipotent differentiation capacity. In other words these cells not only define mesenchyml stromal cells (adipogenic, chondrogenic, osteogenic differentiation), but also other capacities, including differentiation to cells derived from of all three germ layers, i.e. 1. endoderm (e.g. angiogenesis—e.g. tube formation, CD31 and VEGFR1 expression), 2. mesoderm (e.g. myogenesis—e.g. spectrin, desmin expression) and 3. ectoderm (e.g. neurogenesis—e.g. Tuj1 expression).

Moreover, the in vitro manufactured cells possess corneal epithelial differentiation capacity (e.g. KRT12 expression), which can be used to treat limbal stem cell deficiency and other corneal disorders in vivo. The term “possess corneal epithelial differentiation capacity” refers to an ability of a cell to express a protein. The stem cells might not express KRT12 or other proteins. However, the cells have the ability to differentiate into cells that express any of these proteins. Importantly, the presence of KRT12 in differentiated versions of this synthetic cell population provides these cells with the unique capability to treat corneal disorders. This factor is often missing from populations of stem cells isolated from human tissue. It has been proposed that in order to treat corneal disease with these isolated human cells, KRT12 should be added to the cells.

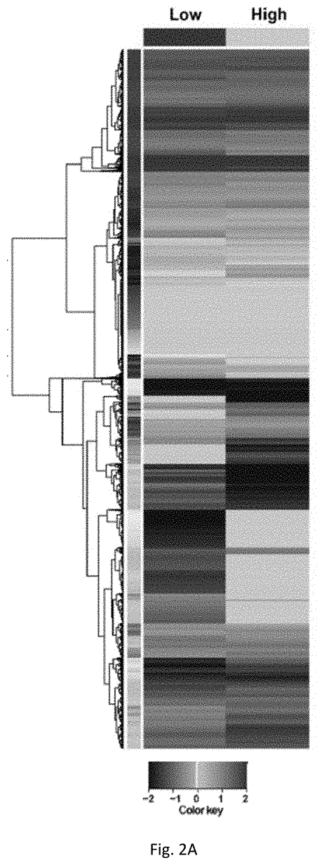

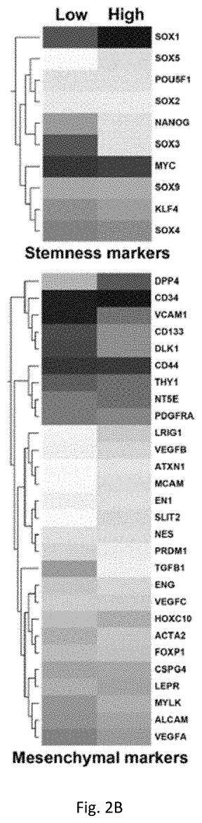

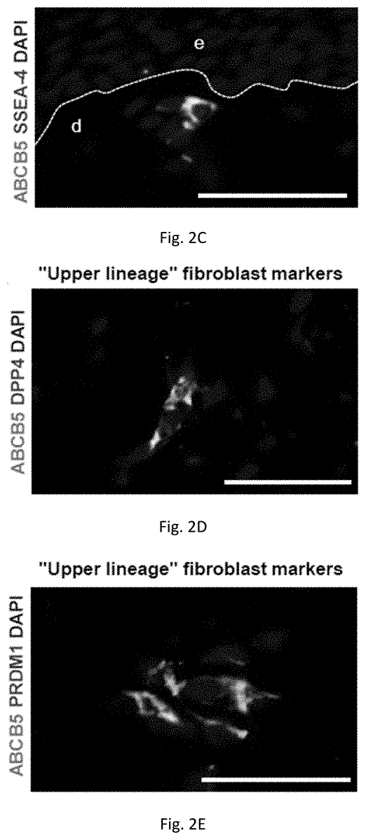

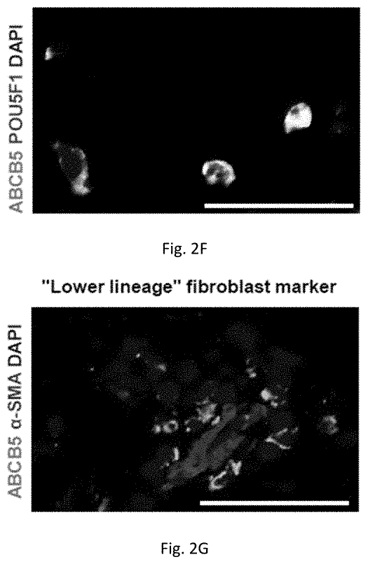

The synthetic cells of the invention also have distinct gene expression profiles relative to primary stem cells isolated from human tissue. As shown in the Examples presented herein, including in FIG. 2, the populations of synthetic cells (also referred to as ABCB5+ cells isolated from high passages) are different from the primary cells (those derived from low passage cultures that contain the native ABCB5+ cells found in the living organism). For example, certain stem cell markers are increased in high passage cells, e.g. SOX2, NANOG and SOX3, while certain mesenchymal stromal differentiation markers are decreased, e.g. MCAM, CRIG1 and ATXN1. The expression of selected stemness markers such as SSEA-4, DPP4 (CD26), PRDM1 (BLIMP1) and POU5F1 (OCT-4) in ABCB5+ cells in human skin at protein level was confirmed by immunostaining. While the expression of lower fibroblast lineage marker α-smooth muscle actin (α-SMA) was absent in ABCB5+ cells of human skin. These data support the finding that these late passage synthetic cells maintain pluripotent properties of ABCB5+ cells, and even have enhanced properties relative to the original cells.

The methods described herein result in highly pure synthetic cell populations. In some preferred embodiments, 100% of the cells are synthetic, with 0% of the cells originating from the human tissue. In some embodiments, the process of the invention allows for up to 16 passages, which equals 25 cell doublings. The percentage of cells synthesized in vitro should therefore be at least the following at each passage, estimated with the following formula:

[1−1/(2ⁿ)]×100%, where n is the doubling number for each passage (i.e. 25 for passage 16, or x/16×25 for passage number x).

As of the 2^{nd} and 3^{rd} passage the structure of the cells begin to change. For instance, the data in gene expression profiling discussed above and presented in the Examples were shown for low passages (2 to 3). Accordingly, a relatively low passage of 3 (with 3/16×25=4.6875 doublings) would result in at least 96.12% of in vitro manufactured or synthetic cells. A high (>10 passage culture) with at least 10/16×25=15.625 doublings would result in at least 99.998% of in vitro manufactured or synthetic cells. A highest passaged cell population tested herein (16 passages) with 25 doublings would result in at least 99.999997% of in vitro manufactured or synthetic cells.

Since stem cells can also divide symmetrically and asymmetrically, the highly passaged cells may reach 100% synthetic cells. Typical passages in the process range from 6 (9.375 doublings) up to 16 passages (25 doublings), i.e. the range of synthetic purity of the product is typically from [1−1/(2⁹0.375)]×100% to [1−1/(2²⁵)]×100%, i.e. from 99.85 to 99.999997%.

### Cell Manufacturing Process

Preparation and processing of the cells takes place in accordance with the guidelines and standards consistent with GMP. The manufacturing process may be performed in a clean room environment. The manufactured cells produced as described herein are cryopreserved and stored in the gas-phase of liquid nitrogen (≤−130° C.).

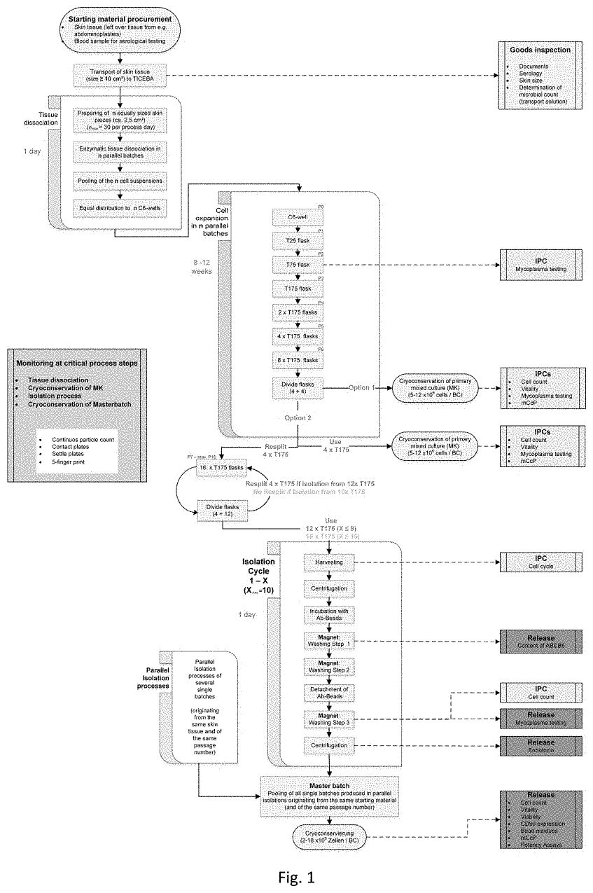

The basic manufacturing process typically involves four steps: Tissue procurement; Processing of the skin tissue; Propagation of the cells; and Isolation of ABCB5-positive cells. The skin tissue may be taken from human surgical specimens such as abdominoplasties (or other medical interventions resulting in left-over skin tissue). A general flow chart depicting the manufacturing steps required to produce the synthetic stem cells disclosed herein starting with skin donor tissue (≥10 cm2) is shown in FIG. 1. In-process and release controls are colored in orange. T25, T75, T175 refer to growth area and associated name of cell culture flasks (cm2). Cryo refers to cryogenic storage of cells in the gas-phase of liquid nitrogen. BC is a barcoded cryo vial. mCcP refers to microbiological control of cellular products. Additionally, other in-process controls (IPCs) may be utilized including Collagenase/TrypZean dissociation of the skin [%], cell morphology, time between passages, confluence, detachment of cells after TrypZean application, incubation times.

ABCB5-positive cells resulting from one isolation (with antibody-coupled magnetic beads) are referred to as “single batch”. Single batches resulting from parallel isolations (originating from the same skin tissue and isolated at the same passage number and time) are pooled (generating a “Masterbatch”) and cryopreserved containing at least 2×10⁶ cells/barcoded cryovial (BC). Parallel to the manufacturing process all steps as well as all lot numbers of used reagents and critical materials are documented in the specific batch documentation. The unique BC-number, unique batch number and the clear allocation of the storage location (in the nitrogen tank) allows for a clear allocation of the produced cell batches. These attributes are documented in batch documentation and additionally in a ‘storage location list’ at the respective nitrogen storage tank.

### Tissue Procurement:

A starting material is leftover skin tissue from surgical procedures such as abdominoplasties or other medical interventions which are conducted at specialized removal centers.

### Processing of the Skin Tissue

The skin is removed from excess subcutaneous fat before its size is determined (skin size needs to be ≥10 cm²). The skin is then cut into equal sections (each around 2.5 cm²). A maximum of 30 pieces can be processed per process day (the remaining pieces are stored in a HTS-FRS biopsy transport solution at +2-+8° C. until processing). Each of two pieces are combined, so in total several preparations can be performed in parallel per process day. For disinfection, the skin pieces are first incubated in aqueous povidone-iodine solution (Braunol®) and then in an alcohol-based povidone-iodine solution (Braunoderm®) at room temperature (RT). Thereafter, the skin tissue is washed 3 times using PBSCa/Mg for each washing step. The skin is dissected using scissors and tweezers. The resulting skin pieces are further dissociated using the enzyme Collagenase: the skin samples are incubated at 37° C. for 1.5-6 h (IPC) in a Collagenase/PBSCa/Mg/Pen/Strep solution. Digestion efficiency after the incubation period needs to be more than 60% (IPC) and is determined visually. The skin-cell solution is filtered, and the residual skin is further incubated using non-animal recombinant trypsin (TrypZean®; Sigma-Aldrich) at 37° C. for 10-60 min (IPC). The filter flow-through as well as the repeatedly filtered TrypZean-treated residual skin (digestion efficiency: >85%, determined visually) (IPC) is washed by centrifugation (500×g, 5 min at RT). After centrifugation, the supernatant is removed, and the cell pellets are resuspended in stem cell medium (HAM's F10 supplemented with 15% FCS, 2 mM L-Glutamine, 0.6 ng/ml bFGF/FGF-2, 6 mM HEPES, 2.8 μg/ml Hydrocortisone, 10 μg/ml Insulin, 1.12 mg/ml Glycose, 6.16 ng/ml PMA, 0.5 μg/ml Amphotericin and 1×Pen/Strep). Cells are pooled, distributed equally on up to 30 wells of C6-cell culture plates and incubated in a cell culture incubator (CO2-content: 3.1%, humidity: 90%; temperature: 37° C.).

### Propagation of the Cells (Mixed Cell Culture)

A mixed cell culture is defined as unsegregated cell culture consisting of ABCB5-positive and ABCB5-negative cells before isolation.

The first assessment of the cell confluence (determined visually by trained employees) takes place 1-4 days (IPC) after cultivation of the primary skin cells in the C6-well. If the confluence is <70% (IPC), culture medium is changed, and cells are further cultivated in the C6-well. This procedure is repeated until cells reach ≥70% confluence (IPC). It should be noted that the primary skin cells are kept in antibiotic/antimycotic containing culture medium only for the initial 4-6 days (IPC). After this initial period, cells are cultivated only in antibiotic-free medium. In addition, the maximum cultivation time in the C6-well is 16 days (IPC). If the cells fail to reach a confluence ≥70% (IPC) within this period, they are discarded.

If the target confluence of ≥70% (IPC) is reached, cells are harvested using TrypZean® and cultured in T25 plates for further expansion. Cell confluence is determined again 1-4 days (IPC) after passaging. If the cell confluence is <70% (IPC), the medium is changed, and cells are further incubated up to 7 days (IPC) total in the T25 vessel (if cell confluence is again <70%, cells are discarded) (IPC). Upon reaching a cell confluence ≥70% within the 7 days, cells are harvested using TrypZean® and cultured in a T75 plate for expansion. At this point, a sample for mycoplasma testing in accordance with 2.6.7. E.P. is taken (IPC). Further cell expansion follows the same scheme.

### MK Cryo-Preservation

Cells were harvested using TrypZean and a cell sample is taken for determination of cell count and vitality. The cell suspension is centrifuged, cells are resuspended in the DMSO-containing cryomedium CS10 (freezing medium containing DMSO). A sample for mycoplasma testing is taken before the cells are transferred into a defined number of barcode labeled cryo tubes (“BCs”), the number depending on the determined cell count. At least 8×10⁶ cells are required for cryopreservation of MK. Minimum one BC (more at higher cell numbers) is filled with 5-12×10⁶ cells (final cell-CS10 solution volume is 1.5 ml). Furthermore, to determine the sterility of the mixed primary culture, a cell sample is taken for testing the mCcP.

### Subcultivation

The residual 4×T175 flasks are used to passage the cells to 16×T175 culture flasks. These 16×T175 flasks are used for isolating ABCB5-positive cells (synthetic stem cells). For the first isolation, the time since the last passage must be between 3-10 days and cells must have reached a certain confluence. In general, for initiating further production steps, the confluence needs to be between 40%-95%.

For the isolation of ABCB5-positive cells 12 of the 16×T175 flasks are used. The cells of the remaining 4×T175 vessels are distributed to 16×T175 flasks as already described to grow cell for the next round of synthetic stem cells isolation until the maximal passage number of 16 is reached or the cell morphology changes (e.g. a more differentiated cell morphology) or the cells become senescence.

### Isolation of ABCB5-Positive Cells (Synthetic Stem Cells)

The isolation process is divided into two parts:
Magnetic isolation of ABCB5-positive cells—generation of single batches of ABCB5-positive cells
Pooling of single batches of one donor with the same passage number (parallel cell isolations originating from the same skin tissue—generation of a master batch

### Magnetic Isolation of ABCB5-Positive Cells

When cells (of 16×T175 flasks) have reached a confluence of 75%-95% the medium of 12×T175 flasks is removed and cells are washed with PBS. Additionally, a sample is taken to determine possible mycoplasma contamination). For harvesting, cells are incubated with Versene® (0.02% EDTA in PBS) for 20-30 min at 37° C. until >90% of the cells are detached from the culture vessel. For this process step Versene is used instead of TrypZean since TrypZean treatment results in the loss of the epitope needed for the antibody-based cell isolation. Cells are diluted by adding PBS to the cell suspension which is then centrifuged at room temperature at 500×g for 5 min. Supernatant is removed and all cells are resuspended in a total of 14 ml HRG (49.5 Vol/% of 5% HSA/49.5 Vol/% Ringer lactate/1 Vol/% of 40% glucose) solution and transferred to a 50-ml reaction tube. A sample is removed and transferred to Quality Control for determination of cell count and vitality and a sample (10⁶ cells) for cell cycle analysis.

400 μl ABCB5-targeting antibody-conjugated magnetic beads are added to the cells and the final volume is adjusted to 16 ml with HRG. The antibody-labeled bead-cell mixture is incubated for 20 min at room temperature using a sample rotator.

29 ml HRG are added to the solution and the sample is incubated on a magnet attracting the magnetic beads to the vessel wall for 4 min. After this incubation period, the supernatant, mainly containing ABCB5 negative or low expressing cells, is carefully removed. The remaining antibody-bead-cell mixture is washed using 45 ml HRG solution. A sample is removed (bead-cell mix) for ABCB5-content determination and transferred to Quality Control (Release parameter).

The remaining solution is incubated on the magnet for additional 4 min. After discarding the supernatant, 3 ml detach solution (TrypZean) are added to enzymatically remove the antibody-labeled beads from the ABCB5 positive cells. This is possible since TrypZean treatment results in the unspecific removal of the antibody-bound epitope (peptide cleavage) and therefore leads to the separation of the antibody-beads from the cells.

After 3 min of incubation at 37° C., 3 ml HRG solution are added to the reaction tube which is again placed on the magnet for 6 min to bind the magnetic beads. The supernatant containing the separated ABCB5 positive cells is then transferred to a fresh 15 ml reaction tube. The 50 ml reaction tube is rinsed twice by adding 3.5 ml HRG solution and magnet incubation for 4 min. The supernatant is then also transferred to the fresh 15 ml tube.

To further purify ABCB5 positive cells from residual beads, they are again held to the magnet for 4 min. The supernatant (13 ml cell suspension) is transferred to a new 15 ml reaction tube and is centrifuged at RT for 5 min and 500×g. The supernatant is discarded, the cell pellet is resuspended in 10 ml HRG solution and again incubated on the magnet for 6 min before the cell suspension is transferred to a new 15 ml reaction tube. Samples for mycoplasma testing (Release parameter) and determination of the cell count of the isolated ABCB5-positive cells (IPC) are taken and transferred to Quality Control. The solution is centrifuged at RT for 5 min and 500×g. Before discarding the supernatant, 100 μl are transferred (with endotoxin-free pipette tips) to an endotoxin-free tube used for endotoxin determination (Release parameter). The remaining supernatant is also carefully removed.

Pooling Step to Generate the Master Batch

A Master Batch (one final batch of synthetic stem cells) consists of single batches that are:
Originating from the same starting material (same Donor)
Isolated in parallel on the same day with the same passage number

The cell pellets of the single batches are resuspended in CryoStor™ CS10. The total amount of CS10 and the associated number of barcode tubes (BCs) depends on the number of available cells. Each BC is filled with 1.5 ml cell suspension in CS10.

Vials are filled at a minimum of 2×10⁶ cells (2-18×10⁶ cells/BC). Before freezing the BCs, one BC is chosen as “Analytic BC for QC” (BC-No. 1) and the following samples are removed and transferred to Quality Control for analytical tests (release testing):
cell count and vitality
viability, CD90 co-expression, bead residues
microbiological control of cellular products (mCcP)

The BC-tubes are frozen to −150° C. with a controlled rate freezer (freezing rate: 1° C./min. until −100° C.; 5° C./min until −150° C.) and are transferred into the quarantine storage tank until their release.

For conducting all three potency assays (tube formation assay, VEGF ELISA and IL-1RA ELISA), the “Analytic BC for QC” is thawed by Quality Control and the cell samples for the assay testing are taken.

In these cases, the cryopreserved mixed culture (MK) can be thawed and used for further cell production. Thus, a large amount of ABCB5-positive cells can be isolated from one single skin tissue resulting in a “Biobank” for clinical use.

The synthetic stem cells produced by these method were determined to have the following specifications:

| Parameter | Test Method | Specification |
|---|---|---|
| Microbiological control | Adapted to 2.6.27 E.P. | No growth |
| cellular products | | |
| Mycoplasma | NAT (2.6.27 E.P.) | Not detectable, <10 |
| | | CFU/ml |
| Total count viable | Flow cytometry | 2-18 × 106 cells |
| cells/BC cryo tube | (2.7.29 E.P.) | |
| Endotoxin level | LAL-test (2.6.14 E.P.) | ≤2 EU/ml |
| Cell vitality | Flow cytometry | ≥90% |
| | (2.7.29 E.P.) | |
| Cell viability | Flow cytometry | ≥90% |
| CD90 surface | Flow cytometry | ≥90% |
| expression | | |
| Bead residues | Flow cytometry | ≤0.5% |
| Content of | Flow cytometry | ≥90% |
| ABCB5-positive cells | | |
| Cell cycle | Flow cytometry | Determined and |
| | | declared |
| Potency Assay | Tube Formation Assay | Successful |
| (angiogenic | | differentiation |
| differentiation) | | |
| Potency Assay | ELISA | >46.9 pg/ml VEGF |
| (VEGF secretion | | (supernatant) |
| after hypoxia) | | |
| Potency Assay | ELISA | >125 pg/ml IL-1RA |
| (IL-1RA secretion | | |
| after co-cultivation | | |
| with M1-polarized | | |
| macrophages) | | |

The analytical procedures used to assess these specifications are described in more detail below.

1. mCcP (Microbiological Control of Cellular Products)

For the sterility testing of the product synthetic stem cells the method “mCcP” is used. The sampling and probing is done within clean room facilities under laminar flow hoods by trained employees of the manufacturing department. The incubation and analysis are done by trained employees of the department.

### Description of the Procedure:

1% of the total end volume of the product is used for mCcP testing. 2×15 μl for mCcP testing are taken directly from each cryo vial (1.5 ml) of each isolated synthetic stem cells batch.

The mCcP is performed with the BacT/Alert 3D 60 system (Biomerieux). The BacT/Alert 3D 60 system consists of 2 modules, one controller module and one incubator module with capacity to simultaneously incubate and detect contamination within 60 individual samples. The media containing bottles are placed into the incubator module, which is equipped with a shaking mechanism.

The following culture media (provided in bottles) are used:
BPA (aerobic): 40 ml Supplemented TSB Atmosphere of CO2 in oxygen
BPN (anaerobic): 40 ml Supplemented TSB Atmosphere of CO2 in nitrogen

For mCcP testing, 15 μl of the testing material is transferred into a BPN or BPA flask, respectively.

Since the sample size is very low, it is diluted to a volume of 4 ml with a NaCl-pepton buffer solution. For mCcP-testing 4 ml sample solution (containing 15 μl cell/CS10 solution) are injected into a BPA and a BPN bottle using sterile syringes. Specialized Liquid Emulsion Sensors (LES) at the bottom of each culture bottle visibly change color (from gray to yellow) when the pH changes due to the rise in CO2 as it is produced by microorganisms. BacT/ALERT® 3D instruments measure the color changes every ten minutes and analyze the changes. Once growth is detected, the system alarms both audibly and visually and the sample data is recorded.

The sensitive procedure allows a precise statement within 7 days. After this time a seeding onto solid culture medium is done for all negative probes. Furthermore, all positive samples are generally seeded onto solid culture medium at the moment of detection.

Planned Proceeding for Sampling

For the planned sampling procedure sample size calculation for the mCcP is based on the total batch volume instead of the volume of the cryovial and the entire sample volume is taken from one dedicated unit.

At least 1% of the total end volume of the product is used for mCcP testing. This means either 100 μl (total product volume ≤10 ml) or 1% of the total product volume (volume >10 ml) for mCcP testing are taken directly from the “Analytic BC for QC” (BC-No.1) of the synthetic stem cells batch.

The low sample size is diluted to a volume of 4 ml with a NaCl-pepton buffer solution (according to E.P.). For mCcP-testing 4 ml sample solution (containing 100 μl-300 μl cell/CS10 solution) are injected into a BPA and a BPN bottle using sterile syringes.

After the incubation time, no microbiological growth may be detected. If this acceptance criterion is met then the product fulfills the requirement “no growth” of the specification parameter “microbiological growth of cellular products.”

2. Mycoplasma Testing

For mycoplasma testing of the product synthetic stem cells the qPCR method is performed. For quantitative Realtime-PCR based Mycoplasma testing the Microsart® ATMP Mycoplasma Kit (Minerva Biolabs) is used which was validated by the manufacturer (Minerva Biolabs) with respect to detection limit for all listed mycoplasma-species, specificity and robustness for cell cultures and autologous cell transplants. The mycoplasma detection is based on the amplification and detection of a highly-conserved RNA-operon, the 16S rRNA-coding region within the mycoplasma genome.

For the performance of the mycoplasma qPCR the StepOne™ Real-Time PCR-system from Life technologies is used.

For mycoplasma testing 200 μl cell suspension is taken after isolation of ABCB5-positive cells during the last washing step on the magnet prior pooling and cryopreservation of the cells. After centrifugation (13000 rpm, 15 min) of the sample the pellet is suspended in 200 μl Tris buffer.

The sample is spiked with internal control DNA and genomic DNA is isolated using the Microsart AMP Extraction Kit. 10 μl of the isolated DNA are used for the qPCR, which is performed in 48-well plates. The qPCR includes positive and negative controls (provided by the Microsart® ATMP Mykoplasma Kit) as well as an internal isolation control and 10 CFU™ Sensitivity Standards for the mycoplasma species Mycoplasma orale (MO), Mycoplasma fermentans (MF) and Mycoplasma pneumoniae (MP) as standards for sensitivity.

The analysis of the qPCR results is done. The negative control must show a Ct-value ≥40, the positive control as well as the sensitivity standards must show Ct-values <40. The sample taken from the process is mycoplasma positive with a Ct-value <40 and mycoplasma negative with a Ct-value ≥40.

In the tested cell suspension, no amplification of mycoplasma DNA may be detectable (detection limit 10 CFU/ml). If this acceptance criterion is met (for all single batches of a master batch) then the product fulfils the requirement “not detectable, <10 CFU/ml” of the specification parameter “Mycoplasma”.

3. Endotoxin Level

For the quantitative determination of the Endotoxin level the chromogenic-kinetic LAL-test is used. This is a quantitative photometric method. The measurement is performed using the Endosafe®-PTS™ and matching LAL-cartridges (both from Charles River Laboratories). The Endosafe®-PTS Cartridges are FDA-licensed as LAL-test method for In-process controls and product end controls of pharmacological products. The endotoxin test is performed with an incubation temperature of 37° C.±1° C., which is recommended by the manufacturer of the lysate. Each cartridge contains a defined amount of a FDA-approved LAL-reagent, chromogenic substrate and an Endotoxin standard control (CSE).

After the isolation of ABCB5-positive cells, separation from the antibody-bead complexes and centrifugation of the cells, 100 μl supernatant is taken for Endotoxin testing and diluted 1:10 with LAL reagent water (LRW-water). For each measurement 25 μl sample are pipetted into each of the 4 sample reservoirs of the LAL-cartridge (inserted in the Endosafe®-PTS™). The PTS™ reader mixes the samples with LAL-reagent (sample channels) or with LAL-reagent and the positive control (spike channels) in 2 channels each. After incubation and addition of the chromogenic substrate the optical density of each well is analyzed kinetically and measured based on the internal batch-specific standard curve.

The evaluation of the duplicate determination is done by calculating the variation of the response time between the two measurements. If the variation of the response time of the duplicate measurements is less than 25 percent, then the endotoxin measurement is regarded as valid.

According to the specification an Endotoxin level ≤2 EU/ml must be achieved by the measured sample (for all single batches of a master batch).

4. Cell Count and Cell Vitality

An automated method for the determination of cell count and cell vitality (is used by using Flow Cytometry. Flow Cytometry (BD Accuri™ C6 Flow Cytometer) provides a rapid and reliable method to quantify live cells in a cell suspension. One method to assess cell vitality is using dye exclusion. Live cells have intact membranes that exclude a variety of dyes that easily penetrate the damaged, permeable membranes of non-viable cells.

Propidium Iodide (PI) is a membrane impermeable dye that is generally excluded from viable cells but can penetrate cell membranes of dying or dead cells. It binds to double stranded DNA by intercalating between the base pairs. PI is excited at 488 nm and, with a relatively large Stokes shift, emits at a maximum wavelength of 617 nm.

The determination of the cell counts as well as vitality is performed after the isolation of synthetic stem cells, directly before their cryopreservation.

For the analysis 10 μl cell suspension are pipetted from the cryo vial into 1.5 ml reaction tubes (containing 80 μl Versene) and handed over to the quality control department. After addition of 10 μl PI solution (1 mg/ml) the total volume is adjusted to 500 μl with Versene and the measurement is performed with the BD Accuri™ C6 Flow Cytometer according to work instruction. Each measurement run is performed with 55 μl sample solution. Cell count and vitality are calculated and documented in the test reports.

The specified acceptance criterion for cell vitality is ≥90%. The specified acceptance criterion for the cell count of each batch of isolated synthetic stem cells is 2×10⁶-18×10⁶ cells/cryo vial.

5. Cell Viability

An automated method for the determination of cell viability is performed by using flow cytometry. To determine viability cells are stained with Calcein-AM (Calcein Acetoxymethylester). Calcein AM is a non-fluorescent, hydrophobic compound that easily permeates intact, live cells. Upon entering the cell, intracellular esterases cleave the acetoxymethyl (AM) ester group producing calcein, a hydrophilic, strongly fluorescent compound that is well-retained in the cell cytoplasm.

Apoptotic and dead cells with compromised cell membranes do not retain Calcein. Calcein is optimally excited at 495 nm and has a peak emission of 515 nm.

The cell viability measurement is performed for the isolated ABCB5-positive cells (synthetic stem cells) immediately prior to cryopreservation of the cells. The cell viability rate provides information about the actual metabolic activity of the isolated cells unlike the cell vitality determination with PI which only discriminates live from dead cells.

For the measurement 100 μl cell suspension (in cryomedium CS10) are taken from the cryo tube, transferred into a 1.5 ml reaction tube containing 1 ml Versene (0.02% EDTA) and handed to Quality Control. Samples may be stored at 2-8° C. for a maximum of 2 h. For sample preparation cells are centrifuged (5 min, 1500 rpm), supernatant is removed and the cell pellet is resuspended in 200 μl Versene. After addition of 2 μl Calcein-AM (1:200 diluted, f.c. 0.1 μM) (and 1 μl CD90-antibody) samples are incubated for 30 min at 37° C. followed by a washing step with 1 ml Versene, centrifugation (5 min, 1500 rpm) and resuspension of the pellet in 200 μl Versene. The measurement of the cell viability is performed with the BD Accuri™ C6 Flow Cytometer. Viability is calculated using the detected calcein fluorescence and documented in the test reports.

The specified acceptance criterion for cell viability is ≥90%.

6. CD-90 Surface Marker

To show that the isolated ABCB5+ cells are indeed stem cells the expression of the surface protein CD90, which is a mesenchymal stem cell marker, is analyzed by Flow Cytometry (BD Accuri™ C6 Flow Cytometer). For the detection of CD90 an Alexa Fluor® 647-conjugated antibody, directed against CD90 is used. Alexa Fluor® 647 dye is a bright, far-red-fluorescent dye that is highly suitable for Flow Cytometry applications with excitation ideally suited for the 594 nm or 633 nm laser lines. For stable signal generation in imaging and Flow Cytometry, Alexa Fluor® 647 dye is pH-insensitive over a wide molar range. Due to the different excitation and emission wave length of Alexa Fluor® 647 and Calcein (see viability testing) the parallel Flow Cytometry analysis of Alexa Fluor® 647 CD90 and Calcein-AP can be performed.

For the measurement 100 μl cell suspension (in cryomedium CS10) are taken from the cryo tube, transferred into a 1.5 ml reaction tube containing 1 ml Versene (0.02% EDTA) and handed to Quality Control. Samples may be stored at 2-8° C. for a maximum of 2 h. For sample preparation cells are centrifuged (5 min, 1500 rpm), supernatant is removed and the cell pellet is resuspended in 200 μl Versene. After addition of 1 μl CD90− Alexa Fluor® 647 antibody (1:200) and 2 μl Calcein-AM (1:200 diluted, f.c. 0.1 μM) samples are incubated for 30 min. at 37° C. followed by a washing step with 1 ml Versene, centrifugation (5 min, 1500 rpm) and resuspension of the pellet in 200 μl Versene. The measurement of CD-90 expression with the BD Accuri™ C6 Flow Cytometer is performed. CD90+ cells are detected by their high Alexa Fluor® 647 fluorescence, their content is calculated and documented in the test reports.

The specified acceptance criterion is ≥90% CD90 positive cells.

7. Bead Residues

To check whether the isolated synthetic stem cells have been efficiently and completely separated from the ABCB5-antibody-beads by the detach solution, cells are tested for bead residues. This analytical method is also performed with Flow Cytometry in parallel to viability and CD90 expression testing.

The isolated ABCB5-positive cells are treated with TrypZean whose enzymatic activity causes the complete cleavage of the mAb-binding site on an extracellular loop of the ABCB5 protein. Insufficient detaching of beads or washing of the cells could lead to residual beads in the isolated synthetic stem cells and therefore must be analyzed.

For the visualization/detection of residual beads by Flow Cytometry the BD Accuri™ C6 is used. Before the first analysis a gate was set in the FSC/SSA-Dot Plot using a cell-free ABCB5− bead solution to visualize bead residues. Since it cannot be excluded that cells are also counted/detected in that gate, the analysis is combined with the Calcein staining of the viability testing. For the analysis, only events that lie in the bead gate and are Calcein negative are considered. Thus, viable cells are excluded from the analysis and only beads are counted.

The sample preparation and the measurement with the BD Accuri™ C6 Flow Cytometer is performed as already described in “Cell viability” and “CD90-surface marker” according to work instruction. The proportion of residual beads is calculated and documented in the test reports.

The specified acceptance criterion is ≤0.5% residual beads in synthetic stem cells.

8. ABCB5 Content Determination After the isolation of synthetic stem cells the actual content of ABCB5-positive cells is determined by flow cytometry.

ABCB5-positive cells are detected by using a donkey α-mouse Alexa-647 antibody. This secondary antibody is directed against the monoclonal α-ABCB5 antibody. Additionally, the 2nd antibody is coupled to the fluorochrome Alexa-647 which allows detection with Flow Cytometry. Thereby, the emitted fluorescence directly correlates with the number of bound antibodies but not with the real amount of antibody bound ABCB5-positive cells as also free/un-bound bead-antibody complexes are detected. To obtain the actual number of ABCB5-positive cells, an additional stain with Calcein-AM is performed which allows the discrimination of cells (viable) and bead-antibody complexes (non-viable). By considering only Calcein-positive events for the analysis free bead-antibody complexes are excluded.

Since the detachment of the magnetic beads from the cells with TrypZean leads to the loss of the ABCB5 protein on the cell surface, the detection of ABCB5 with an antibody is not possible after the detachment. Therefore, a 200 μl sample for content determination is taken after addition of the magnetic beads, incubation and magnetic separation but before addition of TrypZean. The cells, still bound to the magnetic antibody-coupled beads, are handed over to quality control and are either directly used for analyzing or stored at 2-8° C. for max. 2 h. After centrifugation cells are resuspended in 200 μl 2nd antibody-solution (donkey α-mouse Alexa 647, diluted 1:500 with Versene) and 7 μl calcein-AM and incubated for 20-30 minutes at 37° C. Cells are centrifuged, washed with Versene and finally resuspended for analyzing in 200 μl Versene.

The measurement of the ABCB5 content with the BD Accuri™ C6 Flow Cytometer is performed according to work instruction. By gating only cells with high calcein fluorescence unbound bead-antibody complexes are excluded from the analysis. The proportion of ABCB5 positive cells is calculated from the Alexa-647 fluorescence of the secondary antibody.

The specified acceptance criterion for the content of ABCB5-positive cells after isolation of synthetic stem cells is ≥90% (for each single batch of a master batch).

9. Potency Assay 1: Angiogenic Differentiation (Tube Formation Assay)

An important criterion for the release of synthetic stem cells is the potency of the cells to trans-differentiate. Within the process, it is tested whether synthetic stem cells can undergo angiogenic differentiation. The differentiation potential/capacity is tested using the so-called Tube Formation Assay, one of the most widely used in vitro assays for measuring angiogenesis. With this fast assay the capacity of cells to build 3-dimensional structures (tube formation) in the presence of an extracellular matrix, is tested.

For the testing of all three Potency Assays the defined “Analytic BC for QC” is used and thawed. The differentiation assay is performed according work instruction. For the Tube Formation Assay 1×105 and 1.5×105 cells are seeded (in stem cell medium) in two wells of a 24-well plate (coated with ECM matrix) and incubated for 19 h-22 h in the CO2-incubator. Pictures are taken under the microscope (40× magnification) and saved for the analysis.

The specified acceptance criterion for the Potency Assay is the formation of tubes (qualitative analysis) for at least one of the two tested cell concentrations.

10. Potency Assay 2: VEGF Secretion after Hypoxia

The VEGF secretion of the isolated cells after hypoxic cultivation serves as second Potency Assay. With this method, the ability of the ABCB5-positive cells to enhance angiogenesis via paracrine factors is tested.

For the testing the defined “Analytic BC for QC” is used and thawed. For the Assay 3×105 cells are seeded (in stem cell medium) into a cell culture dish (35×10 mm) and cultured under hypoxic conditions (1% 02 in hypoxia chamber) for 48 h (±2 h) at 37° C. The supernatant is collected and used for the VEGF ELISA.

The specified acceptance criterion is >46.9 pg/ml VEGF in the cell supernatant after hypoxic cultivation based on validation data.

11. Potency Assay 3: IL-IRA Secretion after Co-Cultivation with M1-Polarized Macrophages

The determination of IL-1RA secretion after co-cultivation with M1-polarized macrophages and stimulation of an inflammatory milieu shall demonstrate the immunomodulatory ability of ABCB5-positive cells.

At the beginning of the assay THP-1 cells are differentiated to macrophages (Mφ) by addition of PMA (150 nmol/ml) to the cell culture medium. After 48 h macrophages are co-cultivated with ABCB5-positive cells (synthetic stem cells). Therefore, the defined “Analytic BC for QC” is used and thawed. In two wells of a 24-well plate 2×10⁴ ABCB5-positive cells are co-cultivated with 1×10⁵ macrophages for 48 h. In one well an inflammatory milieu is stimulated by addition of 50 IU/ml IFN-g at the start of the co-cultivation. The stimulation is repeated after 24 h of co-cultivation by adding 20 ng/ml LPS and again 50 IU/ml IFN-g. After 2 days of co-cultivation supernatants are collected and used for the IL-1RA ELISA.

The specified acceptance criterion is the secretion of >125 pg/ml IL-1RA after co-cultivation with macrophages based on validation data (and stimulation of an inflammatory milieu).

The synthetic ABCB5+ stem cells of the invention may be used for many different therapeutic purposes. For instance, the synthetic cells may be used for syngeneic transplants cutaneous wound healing, allogeneic transplants, peripheral arterial occlusive disease—PAOD, acute-on-chronic liver failure—AOCLF, epidermolysis bullosa—EB and many other diseases. For instance, based on newly demonstrated KRT12+ corneal differentiation capacity, for treatment of limbal stem cell deficiency (LSCD) and other corneal disorders (similar to the limbal ABCB5+ stem cells already in clinical trials as allografts, but with the advantage that the ABCB5+ skin stem cells could be used as autologous patient-syngeneic grafts in LSCD or corneal disorders upon isolation from patient skin, avoiding transplant rejection).

Treatment of inflammatory- and or immunity-caused disorders that involve ILlbeta and are responsive to IL-1RA, as outlined in the Dinarello et al Nat Rev Drug Discov. 2012 paper, or treatment of disorders driven by TNF-alpha (e.g. rheumatoid arthritis) or IL-12/IL-23p40 (e.g psoriasis), or diseases that are amenable to IL-10/regulatory T cell treatment (e.g. transplant rejection) are also envisioned. The potential applications for inflammation-driven disease processes is very large, and includes, for example, cardiovascular disease, ischemic stroke, Alzheimer disease and aging. Similarly, immune disorders such as transplant rejection or graft-versus-host disease, should be amenable to treatment with this cellular therapeutic.

Further treatment of diseases that are based on the neurogenic and myogenic differentiation capacity of this synthetic cellular preparation would be stroke or other CNS disorders that depend on tissue repair for improvement, or musculoskeletal disorders, including e.g. genetic muscular dystrophies, that depend on muscle repair.

The cell composition is also envisioned to be useful in further improvements, including gene transfections to induce expression in the ABCB5+ stem cells for example tissue-specific homing factors to target them to specific tissues, of secreted molecules involved in tissue remodeling, and of growth factors, cytokines, hormones and neurotransmitters that may be dysregulated in a patient. Additionally, corrected genes may be transfected to allow stem cell-based repair of genetic diseases in which particular genes are defective (e.g. COL7A in RDEB), or defective genes in ABCB5+ stem cells may be corrected by various gene editing technologies prior to transplantation to syngeneic patients.

Additionally, these cells may be used as a composition for cellular reprogramming by pluripotency or progenitor genes. For example, we have demonstrated that these cells are more easily reprogrammable to iPSC than ABCB5-cells. Moreover, PAX6 overexpression in these cells can further improve their corneal differentiation capacity, as has been shown for other skin progenitors.

Due to their capacity to engraft and release wound healing promoting factors, profound interest has developed in advanced MSC-based therapies for patients suffering from acute and chronic wounds. To date, 1-2% of the population in developed countries suffer from a non-healing wound and the incidence of chronic wounds is estimated to increase due to the world-wide increase in elderly, obese and diabetic patients [4]. One major hurdle still hampering the successful implementation of large scale MSC-based therapies in clinical practice is the lack of a cell surface marker that reliably allows to enrich and expand MSCs for reproducible paracrine efficacy and potency.

Though different in etiology, chronic wounds share the common feature of persistent high numbers of over-activated pro-inflammatory M1 macrophages [7, 8] with enhanced release of TNFα and other pro-inflammatory cytokines. These pro-inflammatory cytokines, along with proteases and reactive oxygen species, lead to tissue breakdown and the installment of a senescence program in resident wound site fibroblasts, thus perpetuating a non-healing state of these wounds. Iron accumulation was previously identified in macrophages residing in chronic venous leg ulcers as a consequence of persistent extravasation of red blood cells at the wound site due to increased blood pressure and venous valve insufficiency. Iron overloaded macrophages in these wounds fail to switch from their pro-inflammatory M1 state to anti-inflammatory M2 macrophages required for tissue remodeling and restoration [7]. M2 macrophages show a lower inflammatory cytokine release as opposed to their M1 counterparts and produce growth factors and metabolites that stimulate tissue repair and wound healing [9]. Conversely, effector molecules like TNFα and IL-1β, among others released by M1 macrophages, maintain a vicious cycle of autocrine recruitment and constant activation of M1 macrophages, thus virtually locking wounds in a non-healing state of persistent inflammation [7, 8].

The involvement of paracrine mechanisms employed by ABCB5⁺-derived MSCs to counteract persisting inflammation and to switch the prevailing M1 macrophages towards tissue repair promoting M2 macrophages, a prerequisite for healing of chronic wounds, were specifically addressed.

To exclude any engraftment or cell fusion effects, a xenotransplant model was purposely used with local injection of human ABCB5⁺-derived MSCs into chronic wounds of the iron overload murine model, closely mirroring the major pathogenic aspect of unrestrained M1 macrophage activation in human chronic wounds [7]. Clinical grade approved ABCB5⁺ MSC preparations have been employed with documented clonal tri-lineage differentiation capacity, enhanced clonal growth and TNFα suppressing activity in vitro as valuable predictors for successful treatment of chronic wounds in vivo. It was found that ABCB5+-derived MSCs injected into iron overload wounds enhanced release of the paracrine IL-1 receptor antagonist (IL-IRA) and, indeed, switched the prevailing M1 pro-inflammatory macrophage phenotype excessively increased in chronic iron overload murine wounds to an anti-inflammatory M2 macrophage promoting overall wound healing. The causal role of the paracrine release of IL-1RA from injected ABCB5+-derived MSCs was supported by the findings that injection of human recombinant IL-1RA accelerated wound healing, while injection of IL-IRA silenced ABCB5+-derived MSCs did not. Notably, these data are recapitulated in humanized NOD-scid IL2rγ^{null} (NSG) mice, with a shift from human pro-inflammatory M1 to anti-inflammatory M2 macrophages further paving the way for the successful translation of marker-enriched ABCB5+ MSCs therapies into clinical practice for the long-term benefit of the patients.

The synthetic ABCB5+ stem cells are preferably isolated. An “isolated synthetic ABCB5+ stem cell” as used herein refers to a preparation of cells that are placed into conditions other than their natural environment. The term “isolated” does not preclude the later use of these cells thereafter in combinations or mixtures with other cells or in an in vivo environment.

The synthetic ABCB5+ stem cells may be prepared as substantially pure preparations. The term “substantially pure” means that a preparation is substantially free of cells other than ABCB5 positive stem cells. For example, the ABCB5 cells should constitute at least 70 percent of the total cells present with greater percentages, e.g., at least 85, 90, 95 or 99 percent, being preferred. The cells may be packaged in a finished pharmaceutical container such as an injection vial, ampoule, or infusion bag along with any other components that may be desired, e.g., agents for preserving cells, or reducing bacterial growth. The composition should be in unit dosage form.

The synthetic ABCB5+ stem cells are useful in some embodiments for treating immune mediated diseases. Immune mediated diseases are diseases associated with a detrimental immune response, i.e., one that damages tissue. These diseases include but are not limited to transplantation, autoimmune disease, cardiovascular disease, liver disease, kidney disease and neurodegenerative disease.

It has been discovered that synthetic ABCB5+ stem cells can be used in transplantation to ameliorate a response by the immune system such that an immune response to an antigen(s) will be reduced or eliminated. Transplantation is the act or process of transplanting a tissue or an organ from one body or body part to another. The synthetic ABCB5+ stem cells may be autologous to the host (obtained from the same host) or non-autologous such as cells that are allogeneic or syngeneic to the host. Non-autologous cells are derived from someone other than the patient or the donor of the organ. Alternatively the synthetic ABCB5+ stem cells can be obtained from a source that is xenogeneic to the host.

Allogeneic refers to cells that are genetically different although belonging to or obtained from the same species as the host or donor. Thus, an allogeneic human mesenchymal stem cell is a mesenchymal stem cell obtained from a human other than the intended recipient of the synthetic ABCB5+ stem cells or the organ donor. Syngeneic refers to cells that are genetically identical or closely related and immunologically compatible to the host or donor, i.e., from individuals or tissues that have identical genotypes. Xenogeneic refers to cells derived or obtained from an organism of a different species than the host or donor.

Thus, the synthetic ABCB5+ stem cells are used to suppress or ameliorate an immune response to a transplant (tissue, organ, cells, etc.) by administering to the transplant recipient synthetic ABCB5+ stem cells in an amount effective to suppress or ameliorate an immune response against the transplant.

Accordingly, the methods may be achieved by contacting the recipient of donor tissue with synthetic ABCB5+ stem cells. The synthetic ABCB5+ stem cells can be administered to the recipient before or at the same time as the transplant or subsequent to the transplant. When administering the stem cells prior to the transplant, typically stem cells should be administered up to 14 days and preferably up to 7 days prior to surgery. Administration may be repeated on a regular basis thereafter (e.g., once a week).

The synthetic ABCB5+ stem cells can also be administered to the recipient as part of the transplant. For instance, the synthetic ABCB5+ stem cells may be perfused into the organ or tissue before transplantation. Alternatively the tissue may be transplanted and then treated during the surgery.

Treatment of a patient who has received a transplant, in order to reduce the severity of or eliminate a rejection episode against the transplant may also be achieved by administering to the recipient of donor tissue synthetic ABCB5+ stem cells after the donor tissue has been transplanted into the recipient.

Reducing an immune response by donor tissue, organ or cells against a recipient, i.e. graft versus host response may be accomplished by treating the donor tissue, organ or cells with synthetic ABCB5+ stem cells ex vivo prior to transplantation of the tissue, organ or cells into the recipient. The synthetic ABCB5+ stem cells reduce the responsiveness of T cells in the transplant that may be subsequently activated against recipient antigen presenting cells such that the transplant may be introduced into the recipient's (host's) body without the occurrence of, or with a reduction in, an adverse response of the transplant to the host. Thus, what is known as “graft versus host” disease may be averted.

The synthetic ABCB5+ stem cells can be obtained from the recipient or donor, for example, prior to the transplant. The synthetic ABCB5+ stem cells can be isolated and stored frozen until needed. The synthetic ABCB5+ stem cells may also be culture-expanded to desired amounts and stored until needed. Alternatively they may be obtained immediately before use.

The synthetic ABCB5+ stem cells are administered to the recipient in an amount effective to reduce or eliminate an ongoing adverse immune response caused by the donor transplant against the host. The presentation of the synthetic ABCB5+ stem cells to the host undergoing an adverse immune response caused by a transplant inhibits the ongoing response and prevents restimulation of the T cells thereby reducing or eliminating an adverse response by activated T cells to host tissue.

As part of a transplantation procedure the synthetic ABCB5+ stem cells may also be modified to express a molecule to enhance the protective effect, such as a molecule that induces cell death. As described in more detail below, the dermal synthetic ABCB5+ stem cells can be engineered to produce proteins using exogenously added nucleic acids. For instance, the synthetic ABCB5+ stem cells can be used to deliver to the immune system a molecule that induces apoptosis of activated T cells carrying a receptor for the molecule. This results in the deletion of activated T lymphocytes and in the suppression of an unwanted immune response to a transplant. Thus, dermal synthetic ABCB5+ stem cells may be modified to express a cell death molecule. In preferred embodiments of the methods described herein, the synthetic ABCB5+ stem cells express the cell death molecule Fas ligand or TRAIL ligand.

In all cases an effective dose of cells (i.e., a number sufficient to prolong allograft survival should be given to a patient). The number of cells administered should generally be in the range of 1×10⁷-1×10¹⁰ and, in most cases should be between 1×10⁸ and 5×10⁹. Actual dosages and dosing schedules will be determined on a case by case basis by the attending physician using methods that are standard in the art of clinical medicine and taking into account factors such as the patient's age, weight, and physical condition. In cases where a patient is exhibiting signs of transplant rejection, dosages and/or frequency of administration may be increased. The cells will usually be administered by intravenous injection or infusion although methods of implanting cells, e.g. near the site of organ implantation, may be used as well.

The synthetic ABCB5+ stem cells may be administered to a transplant patient either as the sole immunomodulator or as part of a treatment plan that includes other immunomodulators as well. For example, patients may also be given: monoclonal antibodies or other compounds that block the interaction between CD40 and CD40L; inhibitors of lymphocyte activation and subsequent proliferation such as cyclosporine, tacrolimus and rapamycin; or with immunosuppressors that act by other mechanisms such as methotrexate, azathioprine, cyclophosphamide, or anti-inflammatory compounds (e.g., adrenocortical steroids such as dexamethasone and prednisolone).

The dermal synthetic ABCB5+ stem cells of the invention are also useful for treating and preventing autoimmune disease. Autoimmune disease is a class of diseases in which an subject's own antibodies react with host tissue or in which immune effector T cells are autoreactive to endogenous self peptides and cause destruction of tissue. Thus an immune response is mounted against a subject's own antigens, referred to as self antigens. Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus. A “self-antigen” as used herein refers to an antigen of a normal host tissue. Normal host tissue does not include cancer cells.

An example of autoimmune disease is anti-glomerular basement membrane (GBM) disease. GBM disease results from an autoimmune response directed against the noncollagenous domain 1 of the 3 chain of type IV collagen (3(IV)NC1) and causes a rapidly progressive glomerulonephritis (GN) and ultimately renal failure in afflicted patients. As described in the examples below the effectiveness of dermal synthetic ABCB5+ stem cells in a model of GBM has been demonstrated. Autoreactive antibodies recognizing 3(IV)NC1 are considered hallmark of the disease. In addition, 3(IV)NC1-autoreactive T helper (Th)1-mediated cellular immunity has been implicated in its pathogenesis. Anti-GBM disease can be induced experimentally in susceptible mouse strains by immunization with antigen preparations containing recombinant 3(IV)NC1 (r3(IV)NC1), providing for a valuable disease model system to study responses to therapeutic immunomodulation. Antigen-dependent T cell activation and resultant production of interleukin 2 (IL-2) requires two distinct signals: On antigen encounter, naive T cells receive signal 1 through the T cell receptor engagement with the Major Histocompatibility Complex (MHC)-plus antigenic peptide complex on antigen presenting cells (APCs), and signal 2 through positive costimulatory pathways leading to full activation. The critical role of one such positive costimulatory pathway, the interaction of APC-expressed CD40 with its Th ligand CD40L, for disease development in experimental anti-GBM autoimmune GN has recently been demonstrated, and CD40-CD40L pathway blockade has been found to prevent the development of autoimmune GN. Negative T cell costimulatory signals, on the other hand, function to down-regulate immune responses. Regulatory T cells (TREGs) and soluble cytokine mediators, such as interleukin 10 and members of the transforming growth factor β (TGF-β) family, can also attenuate T cell activation and immune effector responses.

Another autoimmune disease is Crohn's disease. Clinical trials for the treatment of Crohn's disease using synthetic ABCB5+ stem cells have been conducted. Crohn's disease is a chronic condition associated with inflammation of the bowels and gastrointestinal tract. Based on the conducted trials the use of synthetic ABCB5+ stem cells for the treatment of Crohn's disease appears promising.

When used in the treatment of an autoimmune disease, the synthetic ABCB5+ stem cells will preferably be administered by intravenous injection and an effective dose will be the amount needed to slow disease progression or alleviate one or more symptoms associated with the disease. For example, in the case of relapsing multiple sclerosis, an effective dose should be at least the amount needed to reduce the frequency or severity of attacks. In the case of rheumatoid arthritis, an effective amount would be at least the number of cells needed to reduce the pain and inflammation experienced by patients. A single unit dose of cells should typically be between 1×10⁷ and 1×10¹⁰ cells and dosing should be repeated at regular intervals (e.g., weekly, monthly etc.) as determined to be appropriate by the attending physician.

The synthetic ABCB5+ stem cells are also useful in the treatment of liver disease. Liver disease includes disease such as hepatitis which result in damage to liver tissue. More generally, the synthetic ABCB5+ stem cells of the present invention can be used for the treatment of hepatic diseases, disorders or conditions including but not limited to: alcoholic liver disease, hepatitis (A, B, C, D, etc.), focal liver lesions, primary hepatocellular carcinoma, large cystic lesions of the liver, focal nodular hyperplasia granulomatous liver disease, hepatic granulomas, hemochromatosis such as hereditary hemochromatosis, iron overload syndromes, acute fatty liver, hyperemesis gravidarum, intercurrent liver disease during pregnancy, intrahepatic cholestasis, liver failure, fulminant hepatic failure, jaundice or asymptomatic hyperbilirubinemia, injury to hepatocytes, Crigler-Najjar syndrome, Wilson's disease, alpha-1-antitrypsin deficiency, Gilbert's syndrome, hyperbilirubinemia, nonalcoholic steatohepatitis, porphyrias, noncirrhotic portal hypertension, noncirrhotic portal hypertension, portal fibrosis, schistosomiasis, primary biliary cirrhosis, Budd-Chiari syndrome, hepatic veno-occlusive disease following bone marrow transplantation, etc.

Stress on the body can trigger adult stem cells to change into specialized cells that migrate to the damaged area and help repair the injury. For example, a damaged liver can send signals to stem cells which respond by creating liver cells for the damaged liver. (Journal of Clinical Investigation 2003 Jul. 15; 112 (2):160-169).

In some embodiments, the invention is directed to treating a neurodegenerative disease, with dermal synthetic ABCB5+ stem cells. In some cases, the invention contemplates the treatment of subjects having neurodegenerative disease, or an injury to nerve cells which may lead to neuro-degeneration. Neuronal cells are predominantly categorized based on their local/regional synaptic connections (e.g., local circuit interneurons vs. long range projection neurons) and receptor sets, and associated second messenger systems. Neuronal cells include both central nervous system (CNS) neurons and peripheral nervous system (PNS) neurons. There are many different neuronal cell types. Examples include, but are not limited to, sensory and sympathetic neurons, cholinergic neurons, dorsal root ganglion neurons, proprioceptive neurons (in the trigeminal mesencephalic nucleus), ciliary ganglion neurons (in the parasympathetic nervous system), etc. A person of ordinary skill in the art will be able to easily identify neuronal cells and distinguish them from non-neuronal cells such as glial cells, typically utilizing cell-morphological characteristics, expression of cell-specific markers, secretion of certain molecules, etc.

“Neurodegenerative disorder” or “neurodegenerative disease” is defined herein as a disorder in which progressive loss of neurons occurs either in the peripheral nervous system or in the central nervous system. Non-limiting examples of neurodegenerative disorders include: (i) chronic neurodegenerative diseases such as familial and sporadic amyotrophic lateral sclerosis (FALS and ALS, respectively), familial and sporadic Parkinson's disease, Huntington's disease, familial and sporadic Alzheimer's disease, multiple sclerosis, olivopontocerebellar atrophy, multiple system atrophy, progressive supranuclear palsy, diffuse Lewy body disease, corticodentatonigral degeneration, progressive familial myoclonic epilepsy, strionigral degeneration, torsion dystonia, familial tremor, Down's Syndrome, Gilles de la Tourette syndrome, Hallervorden-Spatz disease, diabetic peripheral neuropathy, dementia pugilistica, AIDS Dementia, age related dementia, age associated memory impairment, and amyloidosis-related neurodegenerative diseases such as those caused by the prion protein (PrP) which is associated with transmissible spongiform encephalopathy (Creutzfeldt-Jakob disease, Gerstmann-Straussler-Scheinker syndrome, scrapie, and kuru), and those caused by excess cystatin C accumulation (hereditary cystatin C angiopathy); and (ii) acute neurodegenerative disorders such as traumatic brain injury (e.g., surgery-related brain injury), cerebral edema, peripheral nerve damage, spinal cord injury, Leigh's disease, Guillain-Barre syndrome, lysosomal storage disorders such as lipofuscinosis, Alper's disease, vertigo as result of CNS degeneration; pathologies arising with chronic alcohol or drug abuse including, for example, the degeneration of neurons in locus coeruleus and cerebellum; pathologies arising with aging including degeneration of cerebellar neurons and cortical neurons leading to cognitive and motor impairments; and pathologies arising with chronic amphetamine abuse including degeneration of basal ganglia neurons leading to motor impairments; pathological changes resulting from focal trauma such as stroke, focal ischemia, vascular insufficiency, hypoxic-ischemic encephalopathy, hyperglycemia, hypoglycemia or direct trauma; pathologies arising as a negative side-effect of therapeutic drugs and treatments (e.g., degeneration of cingulate and entorhinal cortex neurons in response to anticonvulsant doses of antagonists of the NMDA class of glutamate receptor), and Wernicke-Korsakoff's related dementia. Neurodegenerative diseases affecting sensory neurons include Friedreich's ataxia, diabetes, peripheral neuropathy, and retinal neuronal degeneration. Neurodegenerative diseases of limbic and cortical systems include cerebral amyloidosis, Pick's atrophy, and Retts syndrome. The foregoing examples are not meant to be comprehensive but serve merely as an illustration of the term “neurodegenerative disorder or “neurodegenerative disease”.

Most of the chronic neurodegenerative diseases are typified by onset during the middle adult years and lead to rapid degeneration of specific subsets of neurons within the neural system, ultimately resulting in premature death. Compositions comprising dermal synthetic ABCB5+ stem cells may be administered to a subject to treat neurodegenerative disease alone or in combination with the administration of other therapeutic compounds for the treatment or prevention of these disorders or diseases. Many of these drugs are known in the art. For example, antiparkinsonian agents include but are not limited to Benztropine Mesylate; Biperiden; Biperiden Hydrochloride; Biperiden Lactate; Carmantadine; Ciladopa Hydrochloride; Dopamantine; Ethopropazine Hydrochloride; Lazabemide; Levodopa; Lometraline Hydrochloride; Mofegiline Hydrochloride; Naxagolide Hydrochloride; Pareptide Sulfate; Procyclidine Hydrochloride; Quinelorane Hydrochloride; Ropinirole Hydrochloride; Selegiline Hydrochloride; Tolcapone; Trihexyphenidyl Hydrochloride. Drugs for the treatment of amyotrophic lateral sclerosis include but are not limited to Riluzole. Drugs for the treatment of Paget's disease include but are not limited to Tiludronate Disodium.

The utility of adult stem cells in the treatment of neurodegenerative disease has been described. It has been demonstrated that synthetic ABCB5+ stem cells can change into neuron-like cells in mice that have experienced strokes. Journal of Cell Transplantation Vol. 12, pp. 201-213, 2003. Additionally, stem cells derived from bone marrow developed into neural cells that hold promise to treat patients with Parkinson's disease, amyotrophic lateral sclerosis (ALS), and spinal cord injuries.

The methods of the invention are also useful in the treatment of disorders associated with kidney disease. Synthetic ABCB5+ stem cells previously injected into kidneys have been demonstrated to result in an almost immediate improvement in kidney function and cell renewal. Thus, the dermal synthetic ABCB5+ stem cells of the invention may be administered to a subject having kidney disease alone or in combination with other therapeutics or procedures, such as dialysis, to improve kidney function and cell renewal.

Other diseases which may be treated according to the methods of the invention include diseases of the cornea and lung. Therapies based on the administration of synthetic ABCB5+ stem cells in these tissues have demonstrated positive results. For instance, human synthetic ABCB5+ stem cells have been used to reconstruct damaged corneas. Ma Y et al, Stem Cells, Aug. 18, 2005. Additionally stem cells derived from bone marrow were found to be important for lung repair and protection against lung injury. Rojas, Mauricio, et al., American Journal of Respiratory Cell and Molecular Biology, Vol. 33, pp. 145-152, May 12, 2005. Thus, the dermal synthetic ABCB5+ stem cells of the invention may also be used in the repair of corneal tissue or lung tissue.

The cells of the invention may also be useful in treating disorders associated with a hyper-immune response, such as that referred to as a cytokine storm. Some infectious diseases induce a cytokine profile resembling secondary haemophagocytic lymphohistiocytosis (sHLH). sHLH is a hyperinflammatory syndrome commonly triggered by viral infections and characterized by a fulminant and fatal hypercytokinaemia with multiorgan failure. Cardinal features of sHLH include unremitting fever, cytopenias, and hyperferritinaemia; pulmonary involvement (including ARDS) occurs in approximately 50% of patients. Significant mortality in patients experiencing cytokine storm, and in particular viral induced cytokine storm might be due to virally driven hyperinflammation. In hyperinflammation, immunosuppression is likely to be beneficial.

The cells of the invention are immunomodulatory and represent an effective therapy for the treatment of disorders associated with hyperinflammation including cytokine storm. Often, a cytokine storm or cascade is referred to as being part of a sequence because one cytokine typically leads to the production of multiple other cytokines that can reinforce and amplify the immune response. Disorders associated with hyperinflammation or cytokine storm include viral infections. Infectious viral diseases include but are not limited to, malaria, avian influenza, smallpox, pandemic influenza, adult respiratory distress syndrome (ARDS), severe acute respiratory syndrome (SARS). Certain specific infectious agents include but are not limited to coronavirus, Ebola, Marburg, Crimean-Congo hemorrhagic fever (CCHF), South American hemorrhagic fever, dengue, yellow fever, Rift Valley fever, Omsk hemorrhagic fever virus, Kyasanur Forest, Junin, Machupo, Sabia, Guanarito, Garissa, Ilesha, or Lassa fever viruses.

A method of ameliorating symptoms or treating one or more diseases or conditions that comprise a cytokine storm in a subject comprising the steps of: identifying the subject in need of amelioration of symptoms or treatment of the diseases or conditions triggered by a cytokine storm; and administering one or more pharmaceutical compositions comprising a therapeutically effective amount of an empty liposome, dissolved or dispersed in a suitable aqueous or non-aqueous medium sufficient to reduce the level of cytokines in the subject, wherein the liposome comprises a eutectic mixture comprising a LysoPG, a myristoyl monoglyceride, and a myristic acid.

The severe acute respiratory syndrome corona virus 2 (SARS-CoV-2), the etiologic factor of coronavirus disease 2019 (COVID-19), is an example of an infectious agent that has the potential to cause a cytokine storm in patients. The clinical spectrum of COVID-19 varies from asymptomatic or paucisymptomatic forms to clinical conditions characterized by respiratory failure that necessitates mechanical ventilation and support in an intensive care unit (ICU), to multiorgan and systemic manifestations in terms of sepsis, septic shock, and multiple organ dysfunction syndromes (MODS).

Common clinical features of COVID-19 include cough, sore throat, fever (not in all patients), headache, fatigue, myalgia and breathlessness, making it difficult to distinguish from other respiratory infections. Complications witnessed include acute lung injury, shock, acute kidney injury, liver injury, gastrointestinal symptoms and acute respiratory distress syndrome (ARDS), which represents a significant amount of mortality. Clinically, the immune responses induced by SARS-CoV-2 infection are two phased. During the incubation and non-severe stages, a specific adaptive immune response is required to eliminate the virus and to preclude disease progression to severe stages. However, when a protective immune response is impaired, virus will propagate and massive destruction of the affected tissues will occur, especially in organs that have high ACE2 expression, the virus entry receptor, such as lungs, arteries, heart, kidney, and intestines. The damaged cells induce innate inflammation in the lungs that is largely mediated by proinflammatory macrophages and granulocytes. Lung inflammation is the main cause of life-threatening respiratory disorders at the severe stage. In some cases, chest CT scan show multiple peripheral ground-glass opacities in subpleural regions of both lungs that likely induced both systemic and localized immune response that led to increased inflammation.

Once severe lung damage occurs, efforts should be made to suppress inflammation and to manage the symptoms. Alarmingly, after discharge from hospital, some patients remain/return viral positive and others even relapse. This indicates that a virus-eliminating immune response to SARS-CoV-2 may be difficult to induce at least in some patients and vaccines may not work in these individuals.

Accumulating evidence suggests that a subgroup of patients with severe COVID-19 might have a cytokine storm syndrome since a massive inflammatory cell infiltration and inflammatory cytokines secretion were found in patients' lungs, alveolar epithelial cells and capillary endothelial cells were damaged, causing acute lung injury. COVID-19 disease severity is associated to a cytokine profile resembling sHLH. Similar to sHLH, the cytokine profile of COVID-19 patients is characterized by increased interleukin (IL)-2, IL-7, granulocyte-colony stimulating factor, interferon-γ inducible protein 10, monocyte chemoattractant protein 1, macrophage inflammatory protein 1-α, and tumor necrosis factor-α.

Therefore, since lymphocytopenia is often seen in severe COVID-19 patients, the hypercytokinaemia caused by SARS-CoV-2 virus is likely mediated by leukocytes other than T cells. The ABCB5 cells of the invention can inhibit Tcells and induce Tregs by interaction via PD-1/PDL-1. ABCB5 cells also mediate anti-inflammatory effects through IL-1RA secretion that also results in IL-6 downregulation, interaction with regulatory T cells by PD1 and suppression of neutrophil Granulocytes.

The cells of the invention are useful for migrating to damaged tissues, exerting anti-inflammatory and immunoregulatory functions, promoting the regeneration of damaged tissues and inhibiting tissue fibrosis by interacting with the inflammatory microenvironment. Thus, in some embodiments the ABCB5+ cells disclosed herein are useful for treating hyperimmune disorders, including viral disease, such as SARS-CoV-2 infection. The cells of the invention are also useful in repairing the damaged lung tissue associated with the hyper-inflammatory immune response caused by the SARS-CoV-2 infection.

Other disorders associated with a cytokine storm that may be treated using the immunomodulatory cells of the invention include but are not limited to: sepsis, systemic inflammatory response syndrome (SIRS), cachexia, septic shock syndrome, traumatic brain injury (e.g., cerebral cytokine storm), graft versus host disease (GVHD), or the result of treatment with activated immune cells, e.g., IL-2 activated T cells, T cells activated with anti-CD19 Chimeric Antigen Receptor (CAR) T cells.

Synthetic ABCB5+ stem cells from sources such as bone marrow have also been used in therapies for the treatment of cardiovascular disease. Bone marrow stem cells can help repair damaged heart muscle by helping the heart develop new, functional tissue. Goodell M A, Jackson K A, Majka S M, Mi T, Wang H, Pocius J, Hartley C J, Majesky M W, Entman M L, Michael L H, Hirschi K K. Stem cell plasticity in muscle and bone marrow. Ann N Y Acad Sci. 2001 June; 938:208-18. Bone marrow stem cells placed in damaged hearts after myocardial infarction improved the hearts' pumping ability by 80%. Nature Medicine Journal September 2003 vol. 9 no. 9: 1195-1201.

Cardiovascular disease refers to a class of diseases that involve the heart and/or blood vessels. While the term technically refers to diseases that affects the heart and/or blood vessels, other organs such as, for example, the lungs, and joints might be affected or involved in the disease. Examples of cardiovascular diseases include, but are not limited to atherosclerosis, arteriosclerosis, aneurysms, angina, chronic stable angina pectoris, unstable angina pectoris, myocardial ischemia (MI), acute coronary syndrome, coronary artery disease, stroke, coronary re-stenosis, coronary stent re-stenosis, coronary stent re-thrombosis, revascularization, post myocardial infarction (MI) remodeling (e.g., post MI remodeling of the left ventricle), post MI left ventricular hypertrophy, angioplasty, transient ischemic attack, pulmonary embolism, vascular occlusion, venous thrombosis, arrhythmias, cardiomyopathies, congestive heart failure, congenital heart disease, myocarditis, valve disease, dilated cardiomyopathy, diastolic dysfunction, endocarditis, rheumatic fever, hypertension (high blood pressure), hypertrophic cardiomyopathy, aneurysms, and mitral valve prolapse.

Atherosclerosis is a disease of large and medium-sized muscular arteries and is characterized by endothelial dysfunction, vascular inflammation, and the buildup of lipids, cholesterol, calcium, and/or cellular debris within the intimal layer of the blood vessel wall. This buildup results in plaque (atheromatous plaque) formation, vascular remodeling, acute and chronic luminal obstruction, abnormalities of blood flow, and diminished oxygen supply to target organs.

Atherosclerosis may cause two main problems First, the atheromatous plaques may lead to plaque ruptures and stenosis (narrowing) of the artery and, therefore, an insufficient blood supply to the organ it feeds. Alternatively, an aneurysm results. These complications are chronic, slowly progressing and cumulative. Most commonly, plaque(s) suddenly ruptures (“vulnerable plaque”) causing the formation of a thrombus that will rapidly slow or stop blood flow (e.g., for a few minutes) leading to death of the tissues fed by the artery. This event is called an infarction. One of the most common recognized scenarios is called coronary thrombosis of a coronary artery causing myocardial infarction (MI) (commonly known as a heart attack). Another common scenario in very advanced disease is claudication from insufficient blood supply to the legs, typically due to a combination of both stenosis and aneurysmal segments narrowed with clots. Since atherosclerosis is a body wide process, similar events also occur in the arteries to the brain, intestines, kidneys, legs, etc.

Atherosclerosis may begin in adolescence, and is usually found in most major arteries, yet is asymptomatic and not detected by most diagnostic methods during life. It most commonly becomes seriously symptomatic when interfering with the coronary circulation supplying the heart or cerebral circulation supplying the brain, and is considered the most important underlying cause of strokes, heart attacks, various heart diseases including congestive heart failure and most cardiovascular diseases in general. Though any artery in the body can be involved, usually only severe narrowing or obstruction of some arteries, those that supply more critically-important organs are recognized. Obstruction of arteries supplying the heart muscle result in a heart attack. Obstruction of arteries supplying the brain result in a stroke. Atheromatous plaque(s) in the arm or leg arteries producing decreased blood flow cause peripheral artery occlusive disease (PAOD)

Cardiac stress testing is one of the most commonly performed non-invasive testing method for blood flow limitation. It generally detects lumen narrowing of ˜75% or greater. Areas of severe stenosis detectable by angiography, and to a lesser extent “stress testing” have long been the focus of human diagnostic techniques for cardiovascular disease, in general. Most severe events occur in locations with heavy plaque. Plaque rupture can lead to artery lumen occlusion within seconds to minutes, and potential permanent tissue damage and sometimes sudden death.

Various anatomic, physiological and behavioral risk factors for atherosclerosis are known. These risk factors include advanced age, male gender, diabetes, dyslipidemia (elevated serum cholesterol or triglyceride levels), high serum concentration of low density lipoprotein (LDL, “bad cholesterol”), Lipoprotein(a) (a variant of LDL), and/or very low density lipoprotein (VLDL) particles, low serum concentration of functioning high density lipoprotein (HDL, “good cholesterol”) particles, tobacco smoking, hypertension, obesity (e.g., central obesity, also referred to as abdominal or male-type obesity), family history of cardiovascular disease (eg. coronary heart disease or stroke), elevated levels of inflammatory markers (e.g., C-reactive protein (CRP or hs-CRP), sCD40L, sICAM, etc.), elevated serum levels of homocysteine, elevated serum levels of uric acid, and elevated serum fibrinogen concentrations.

The term myocardial infarction (MI) is derived from myocardium (the heart muscle) and infarction (tissue death due to oxygen starvation). MI is a disease state that occurs when the blood supply to a part of the heart is interrupted. Acute MI (AMI) is a type of acute coronary syndrome, which is most frequently (but not always) a manifestation of coronary artery disease. The most common triggering event is the disruption of an atherosclerotic plaque in an epicardial coronary artery, which leads to a clotting cascade, sometimes resulting in total occlusion of the artery. The resulting ischemia or oxygen shortage causes damage and potential death of heart tissue.

Important risk factors for MI or AMI include a previous history of vascular disease such as atherosclerotic coronary heart disease and/or angina, a previous heart attack or stroke, any previous episodes of abnormal heart rhythms or syncope, older ag (e.g., men over 40 and women over 50), tobacco smoking, excessive alcohol consumption, high triglyceride levels, high LDL (“Low-density lipoprotein”) and low HDL (“High density lipoprotein”), diabetes, hypertension, obesity, and stress.

Symptoms of MI or AMI include chest pain, shortness of breath, nausea, vomiting, palpitations, sweating, and anxiety or a feeling of impending doom. Subjects frequently feel suddenly ill. Approximately one third of all myocardial infarctions are silent, without chest pain or other symptoms.

A subject suspected of having a MI receives a number of diagnostic tests, such as an electrocardiogram (ECG, EKG), a chest X-ray and blood tests to detect elevated creatine kinase (CK) or troponin levels (markers released by damaged tissues, especially the myocardium). A coronary angiogram allows to visualize narrowing or obstructions on the heart vessels.

Myocardial infarction causes irreversible loss of heart muscle cells leading to a thin fibrotic scar that cannot contribute to heart function. Stem cell therapy provides a possible approach to the treatment of heart failure after myocardial infarction as well as atherosclerosis associated with remodeling. The basic concept of stem cell therapy is to increase the number of functional heart muscle cells by injecting immature heart muscle cells directly into the wall of the damaged heart. Myocardial infarction leads to the loss of cardiomyocytes, followed by pathological remodeling and progression to heart failure. One goal of stem cell therapy is to replace cardiomyocytes lost after ischemia, induce revascularization of the injured region. Another goal is to prevent deleterious pathological remodeling after myocardial infarction and associated with atherosclerosis. Autologous or allogeneic synthetic ABCB5+ stem cells are considered to be one of the potential cell sources for stem cell therapy. Thus, the dermal synthetic ABCB5+ stem cells of the invention may be used in the treatment of cardiovascular diseases.

Another use for the dermal synthetic ABCB5+ stem cells of the invention is in tissue regeneration. In this aspect of the invention, the ABCB5 positive cells are used to generate tissue by induction of differentiation. Isolated and purified synthetic ABCB5+ stem cells can be grown in an undifferentiated state through mitotic expansion in a specific medium. These cells can then be harvested and activated to differentiate into bone, cartilage, and various other types of connective tissue by a number of factors, including mechanical, cellular, and biochemical stimuli. Human synthetic ABCB5+ stem cells possess the potential to differentiate into cells such as osteoblasts and chondrocytes, which produce a wide variety of mesenchymal tissue cells, as well as tendon, ligament and dermis, and this potential is retained after isolation and for several population expansions in culture. Thus, by being able to isolate, purify, greatly multiply, and then activate synthetic ABCB5+ stem cells to differentiate into the specific types of mesenchymal cells desired, such as skeletal and connective tissues such as bone, cartilage, tendon, ligament, muscle, and adipose, a process exists for treating skeletal and other connective tissue disorders. The term connective tissue is used herein to include the tissues of the body that support the specialized elements, and includes bone, cartilage, ligament, tendon, stroma, muscle and adipose tissue.

The methods and devices of the invention utilize isolated dermal mesenchymal progenitor cells which, under certain conditions, can be induced to differentiate into and produce different types of desired connective tissue, such as into bone or cartilage forming cells.

In another aspect, the present invention relates to a method for repairing connective tissue damage. The method comprises the steps of applying the dermal mesenchymal stem to an area of connective tissue damage under conditions suitable for differentiating the cells into the type of connective tissue necessary for repair.

The term “connective tissue defects” refers to defects that include any damage or irregularity compared to normal connective tissue which may occur due to trauma, disease, age, birth defect, surgical intervention, etc. Connective tissue defects also refers to non-damaged areas in which bone formation is solely desired, for example, for cosmetic augmentation.

The dermal synthetic ABCB5+ stem cells may be administered directly to a subject by any known mode of administration or may be seeded onto a matrix or implant. Matrices or implants include polymeric matrices such as fibrous or hydrogel based devices. Two types of matrices are commonly used to support the synthetic ABCB5+ stem cells as they differentiate into cartilage or bone. One form of matrix is a polymeric mesh or sponge; the other is a polymeric hydrogel. The matrix may be biodegradable or nonbiodegradeable. The term biodegradable, as used herein, means a polymer that dissolves or degrades within a period that is acceptable in the desired application, less than about six months and most preferably less than about twelve weeks, once exposed to a physiological solution of pH 6-8 having a temperature of between about 25° C. and 38° C. A matrix may be biodegradable over a time period, for instance, of less than a year, more preferably less than six months, most preferably over two to ten weeks.

Fibrous matrices can be manufactured or constructed using commercially available materials. The matrices are typically formed of a natural or a synthetic polymer. Biodegradable polymers are preferred, so that the newly formed cartilage can maintain itself and function normally under the load-bearing present at synovial joints. Polymers that degrade within one to twenty-four weeks are preferable. Synthetic polymers are preferred because their degradation rate can be more accurately determined and they have more lot to lot consistency and less immunogenicity than natural polymers. Natural polymers that can be used include proteins such as collagen, albumin, and fibrin; and polysaccharides such as alginate and polymers of hyaluronic acid. Synthetic polymers include both biodegradable and non-biodegradable polymers. Examples of biodegradable polymers include polymers of hydroxy acids such as polylactic acid (PLA), polyglycolic acid (PGA), and polylactic acid-glycolic acid (PLGA), polyorthoesters, polyanhydrides, polyphosphazenes, and combinations thereof. Non-biodegradable polymers include polyacrylates, polymethacrylates, ethylene vinyl acetate, and polyvinyl alcohols. These should be avoided since their presence in the cartilage will inevitably lead to mechanical damage and breakdown of the cartilage.

In some embodiment, the polymers form fibers which are intertwined, woven, or meshed to form a matrix having an interstitial spacing of between 100 and 300 microns. Meshes of polyglycolic acid that can be used can be obtained from surgical supply companies such as Ethicon, N.J. Sponges can also be used. As used herein, the term “fibrous” refers to either a intertwined, woven or meshed matrix or a sponge matrix.

The matrix is preferably shaped to fill the defect. In most cases this can be achieved by trimming the polymer fibers with scissors or a knife; alternatively, the matrix can be cast from a polymer solution formed by heating or dissolution in a volatile solvent.

The synthetic ABCB5+ stem cells are seeded onto the matrix by application of a cell suspension to the matrix. This can be accomplished by soaking the matrix in a cell culture container, or injection or other direct application of the cells to the matrix.

The matrix seeded with cells is implanted at the site of the defect using standard surgical techniques. The matrix can be seeded and cultured in vitro prior to implantation, seeded and immediately implanted, or implanted and then seeded with cells. In the preferred embodiment, cells are seeded onto and into the matrix and cultured in vitro for between approximately sixteen hours and two weeks. It is only critical that the cells be attached to the matrix. Two weeks is a preferred time for culture of the cells, although it can be longer. Cell density at the time of seeding or implantation should be approximately 25,000 cells/mm³.

Polymers that can form ionic or covalently crosslinked hydrogels which are malleable are used to encapsulate cells. For example, a hydrogel is produced by cross-linking the anionic salt of polymer such as alginic acid, a carbohydrate polymer isolated from seaweed, with calcium cations, whose strength increases with either increasing concentrations of calcium ions or alginate. The alginate solution is mixed with the cells to be implanted to form an alginate suspension. Then the suspension is injected directly into a patient prior to hardening of the suspension. The suspension then hardens over a short period of time due to the presence in vivo of physiological concentrations of calcium ions.

The polymeric material which is mixed with cells for implantation into the body should form a hydrogel. A hydrogel is defined as a substance formed when an organic polymer (natural or synthetic) is cross-linked via covalent, ionic, or hydrogen bonds to create a three-dimensional open-lattice structure which entraps water molecules to form a gel. Examples of materials which can be used to form a hydrogel include polysaccharides such as alginate, polyphosphazines, and polyacrylates, which are crosslinked ionically, or block copolymers such as Pluronics™ or Tetronics™, polyethylene oxide-polypropylene glycol block copolymers which are crosslinked by temperature or pH, respectively. Other materials include proteins such as fibrin, polymers such as polyvinylpyrrolidone, hyaluronic acid and collagen.

In general, these polymers are at least partially soluble in aqueous solutions, such as water, buffered salt solutions, or aqueous alcohol solutions, that have charged side groups, or a monovalent ionic salt thereof. Examples of polymers with acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

Examples of polymers with basic side groups that can be reacted with anions are poly(vinyl amines), poly(vinyl pyridine), poly(vinyl imidazole), and some imino substituted polyphosphazenes. The ammonium or quaternary salt of the polymers can also be formed from the backbone nitrogens or pendant imino groups. Examples of basic side groups are amino and imino groups.

Alginate can be ionically cross-linked with divalent cations, in water, at room temperature, to form a hydrogel matrix. Due to these mild conditions, alginate has been the most commonly used polymer for hybridoma cell encapsulation, as described, for example, in U.S. Pat. No. 4,352,883 to Lim. In the Lim process, an aqueous solution containing the biological materials to be encapsulated is suspended in a solution of a water soluble polymer, the suspension is formed into droplets which are configured into discrete microcapsules by contact with multivalent cations, then the surface of the microcapsules is crosslinked with polyamino acids to form a semipermeable membrane around the encapsulated materials.

Polyphosphazenes are polymers with backbones consisting of nitrogen and phosphorous separated by alternating single and double bonds. The polyphosphazenes suitable for cross-linking have a majority of side chain groups which are acidic and capable of forming salt bridges with di- or trivalent cations. Examples of preferred acidic side groups are carboxylic acid groups and sulfonic acid groups. Polymers can be synthesized that degrade by hydrolysis by incorporating monomers having imidazole, amino acid ester, or glycerol side groups. For example, a polyanionic poly[bis(carboxylatophenoxy)]phosphazene (PCPP) can be synthesized, which is cross-linked with dissolved multivalent cations in aqueous media at room temperature or below to form hydrogel matrices.

The water soluble polymer with charged side groups is ionically crosslinked by reacting the polymer with an aqueous solution containing multivalent ions of the opposite charge, either multivalent cations if the polymer has acidic side groups or multivalent anions if the polymer has basic side groups. The preferred cations for cross-linking of the polymers with acidic side groups to form a hydrogel are divalent and trivalent cations such as copper, calcium, aluminum, magnesium, strontium, barium, zinc, and tin, although di-, tri- or tetra-functional organic cations such as alkylammonium salts. Aqueous solutions of the salts of these cations are added to the polymers to form soft, highly swollen hydrogels and membranes. The higher the concentration of cation, or the higher the valence, the greater the degree of cross-linking of the polymer. Concentrations from as low as 0.005 M have been demonstrated to cross-link the polymer. Higher concentrations are limited by the solubility of the salt.

Preferably the polymer is dissolved in an aqueous solution, preferably a 0.1 M potassium phosphate solution, at physiological pH, to a concentration forming a polymeric hydrogel, for example, for alginate, of between 0.5 to 2% by weight, preferably 1%, alginate. The isolated cells are suspended in the polymer solution to a concentration of between 1 and 10 million cells/ml, most preferably between 10 and 20 million cells/ml.

In an embodiment, the cells are mixed with the hydrogel solution and injected directly into a site where it is desired to implant the cells, prior to hardening of the hydrogel. However, the matrix may also be molded and implanted in one or more different areas of the body to suit a particular application. This application is particularly relevant where a specific structural design is desired or where the area into which the cells are to be implanted lacks specific structure or support to facilitate growth and proliferation of the cells.

The site, or sites, where cells are to be implanted is determined based on individual need, as is the requisite number of cells. One could also apply an external mold to shape the injected solution. Additionally, by controlling the rate of polymerization, it is possible to mold the cell-hydrogel injected implant

Alternatively, the mixture can be injected into a mold, the hydrogel allowed to harden, then the material implanted.

The suspension can be injected via a syringe and needle directly into a specific area wherever a bulking agent is desired, especially soft tissue defects. The suspension can also be injected as a bulking agent for hard tissue defects, such as bone or cartilage defects, either congenital or acquired disease states, or secondary to trauma, burns, or the like. An example of this would be an injection into the area surrounding the skull where a bony deformity exists secondary to trauma. The injection in these instances can be made directly into the needed area with the use of a needle and syringe under local or general anesthesia.

The dermal synthetic ABCB5+ stem cells may be modified to express proteins which are also useful in the therapeutic indications, as described in more detail below. For example, the cells may include a nucleic acid that produces at least one bioactive factor which further induces or accelerates the differentiation of the synthetic ABCB5+ stem cells into a differentiated lineage. In the instance that bone is being formed, the bioactive factor may be a member of the TGF-beta superfamily comprising various tissue growth factors, particularly bone morphogenic proteins, such as at least one selected from the group consisting of BMP-2, BMP-3, BMP-4, BMP-6 and BMP-7.

The cells of the invention may be useful in a method for inducing T cell anergy, in vitro. Induction of T cell anergy involves culturing the dermal synthetic ABCB5+ stem cells in the presence of antigen under conditions sufficient to induce the formation of T cells and/or T cell progenitors and to inhibit activation of the formed T cells and/or T cell progenitors. Anergy is defined as an unresponsive state of T cells (that is they fail to produce IL-2 on restimulation, or proliferate when restimulated)(Zamoyska R, Curr Opin Immunol, 1998, 10(1):82-87; Van Parijs L, et al., Science, 1998, 280(5361):243-248; Schwartz R H, Curr Opin Immunol, 1997, 9(3):351-357; Immunol Rev, 1993, 133:151-76). Anergy can be measured by taking the treated T cells and restimulating them with antigen in the presence of APCs. If the cells are anergic they will not respond to antigen at an appropriate concentration in the context of APCs.

As used herein, a subject is a human, non-human primate, cow, horse, pig, sheep, goat, dog, cat or rodent. Human dermal synthetic ABCB5+ stem cells and human subjects are particularly important embodiments.

In a still further aspect of the invention described herein, synthetic ABCB5+ stem cells may be genetically engineered (or transduced or transfected) with a gene of interest. The transduced cells can be administered to a patient in need thereof, for example to treat genetic disorders or diseases.

The synthetic ABCB5+ stem cells, and progeny thereof, can be genetically altered. Genetic alteration of a synthetic ABCB5+ stem cell includes all transient and stable changes of the cellular genetic material which are created by the addition of exogenous genetic material. Examples of genetic alterations include any gene therapy procedure, such as introduction of a functional gene to replace a mutated or nonexpressed gene, introduction of a vector that encodes a dominant negative gene product, introduction of a vector engineered to express a ribozyme and introduction of a gene that encodes a therapeutic gene product. Natural genetic changes such as the spontaneous rearrangement of a T cell receptor gene without the introduction of any agents are not included in this concept. Exogenous genetic material includes nucleic acids or oligonucleotides, either natural or synthetic, that are introduced into the dermal synthetic ABCB5+ stem cells. The exogenous genetic material may be a copy of that which is naturally present in the cells, or it may not be naturally found in the cells. It typically is at least a portion of a naturally occurring gene which has been placed under operable control of a promoter in a vector construct.

Various techniques may be employed for introducing nucleic acids into cells. Such techniques include transfection of nucleic acid-CaPO₄ precipitates, transfection of nucleic acids associated with DEAE, transfection with a retrovirus including the nucleic acid of interest, liposome mediated transfection, and the like. For certain uses, it is preferred to target the nucleic acid to particular cells. In such instances, a vehicle used for delivering a nucleic acid according to the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. For example, where liposomes are employed to deliver the nucleic acids of the invention, proteins which bind to a surface membrane protein associated with endocytosis may be incorporated into the liposome formulation for targeting and/or to facilitate uptake. Such proteins include proteins or fragments thereof tropic for a particular cell type, antibodies for proteins which undergo internalization in cycling, proteins that target intracellular localization and enhance intracellular half-life, and the like. Polymeric delivery systems also have been used successfully to deliver nucleic acids into cells, as is known by those skilled in the art. Such systems even permit oral delivery of nucleic acids.

One method of introducing exogenous genetic material into the dermal synthetic ABCB5+ stem cells is by transducing the cells using replication-deficient retroviruses. Replication-deficient retroviruses are capable of directing synthesis of all virion proteins, but are incapable of making infectious particles. Accordingly, these genetically altered retroviral vectors have general utility for high-efficiency transduction of genes in cultured cells. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in the art.

The major advantage of using retroviruses is that the viruses insert efficiently a single copy of the gene encoding the therapeutic agent into the host cell genome, thereby permitting the exogenous genetic material to be passed on to the progeny of the cell when it divides. In addition, gene promoter sequences in the LTR region have been reported to enhance expression of an inserted coding sequence in a variety of cell types. The major disadvantages of using a retrovirus expression vector are (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome. Despite these apparent limitations, delivery of a therapeutically effective amount of a therapeutic agent via a retrovirus can be efficacious if the efficiency of transduction is high and/or the number of target cells available for transduction is high.

Yet another viral candidate useful as an expression vector for transformation of dermal synthetic ABCB5+ stem cells is the adenovirus, a double-stranded DNA virus. Like the retrovirus, the adenovirus genome is adaptable for use as an expression vector for gene transduction, i.e., by removing the genetic information that controls production of the virus itself. Because the adenovirus functions usually in an extrachromosomal fashion, the recombinant adenovirus does not have the theoretical problem of insertional mutagenesis. On the other hand, adenoviral transformation of a target dermal mesenchymal stem cell may not result in stable transduction. However, more recently it has been reported that certain adenoviral sequences confer intrachromosomal integration specificity to carrier sequences, and thus result in a stable transduction of the exogenous genetic material.

Thus, as will be apparent to one of ordinary skill in the art, a variety of suitable vectors are available for transferring exogenous genetic material into dermal synthetic ABCB5+ stem cells. The selection of an appropriate vector to deliver a therapeutic agent for a particular condition amenable to gene replacement therapy and the optimization of the conditions for insertion of the selected expression vector into the cell, are within the scope of one of ordinary skill in the art without the need for undue experimentation. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any nontranslated DNA sequence which works contiguous with the coding sequence (in cis) to change the basal transcription level dictated by the promoter. Preferably, the exogenous genetic material is introduced into the dermal mesenchymal stem cell genome immediately downstream from the promoter so that the promoter and coding sequence are operatively linked so as to permit transcription of the coding sequence. A preferred retroviral expression vector includes an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., Proc. Natl. Acad. Sci. USA 88:4626-4630 (1991)), adenosine deaminase, phosphoglycerol kinase (PGK), pyruvate kinase, phosphoglycerol mutase, the actin promoter (Lai et al., Proc. Natl. Acad. Sci. USA 86: 10006-10010 (1989)), and other constitutive promoters known to those of skill in the art. In addition, many viral promoters function constitutively in eukaryotic cells. These include: the early and late promoters of SV40; the long terminal repeats (LTRS) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified dermal mesenchymal stem cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the subject.

In addition to at least one promoter and at least one heterologous nucleic acid encoding the therapeutic agent, the expression vector preferably includes a selection gene, for example, a neomycin resistance gene, for facilitating selection of dermal synthetic ABCB5+ stem cells that have been transfected or transduced with the expression vector. Alternatively, the dermal synthetic ABCB5+ stem cells are transfected with two or more expression vectors, at least one vector containing the gene(s) encoding the therapeutic agent(s), the other vector containing a selection gene. The selection of a suitable promoter, enhancer, selection gene and/or signal sequence is deemed to be within the scope of one of ordinary skill in the art without undue experimentation.

The selection and optimization of a particular expression vector for expressing a specific gene product in an isolated dermal mesenchymal stem cell is accomplished by obtaining the gene, preferably with one or more appropriate control regions (e.g., promoter, insertion sequence); preparing a vector construct comprising the vector into which is inserted the gene; transfecting or transducing cultured dermal synthetic ABCB5+ stem cells in vitro with the vector construct; and determining whether the gene product is present in the cultured cells.

Thus, the present invention makes it possible to genetically engineer dermal synthetic ABCB5+ stem cells in such a manner that they produce polypeptides, hormones and proteins not normally produced in human stem cells in biologically significant amounts or produced in small amounts but in situations in which overproduction would lead to a therapeutic benefit. These products would then be secreted into the bloodstream or other areas of the body, such as the central nervous system. The human stem cells formed in this way can serve as a continuous drug delivery systems to replace present regimens, which require periodic administration (by ingestion, injection, depot infusion etc.) of the needed substance. This invention has applicability in providing hormones, enzymes and drugs to humans, in need of such substances. It is particularly valuable in providing such substances, such as hormones (e.g., parathyroid hormone, insulin), which are needed in sustained doses for extended periods of time.

For example, it can be used to provide continuous delivery of insulin, and, as a result, there would be no need for daily injections of insulin. Genetically engineered human synthetic ABCB5+ stem cells can also be used for the production of clotting factors such as Factor VIII, or for continuous delivery of dystrophin to muscle cells for muscular dystrophy.

Incorporation of genetic material of interest into dermal synthetic ABCB5+ stem cells is particularly valuable in the treatment of inherited and acquired disease. In the case of inherited diseases, this approach is used to provide genetically modified human synthetic ABCB5+ stem cells and other cells which can be used as a metabolic sink. That is, such dermal synthetic ABCB5+ stem cells would serve to degrade a potentially toxic substance. For example, this could be used in treating disorders of amino acid catabolism including the hyperphenylalaninemias, due to a defect in phenylalanine hydroxylase; the homocysteinemias, due to a defect in cystathionine beta-synthase.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### Examples

Here the beneficial effects of a newly identified dermal cell subpopulation expressing the ATP-binding cassette subfamily B member 5 (ABCB5) for the therapy of non-healing wounds were reported. Local administration of dermal ABCB5⁺-derived MSCs attenuated macrophage-dominated inflammation and thereby accelerated healing of full-thickness excisional wounds in the iron overload mouse model mimicking the non-healing state of human venous leg ulcers. The observed beneficial effects were due to interleukin-1 receptor antagonist (IL-1RA) secreted by ABCB5⁺-derived MSCs, which dampened inflammation and shifted the prevalence of unrestrained pro-inflammatory M1 macrophages towards repair promoting anti-inflammatory M2 macrophages at the wound site. The beneficial anti-inflammatory effect of IL-1RA released from ABCB5⁺-derived MSCs on human wound macrophages was conserved in humanized NOD-scid IL2rγ^{null} mice. In conclusion, human dermal ABCB5⁺ cells represent a novel, easy accessible and marker-enriched source of MSCs which holds substantial promise to successfully treat chronic non-healing wounds in humans.

The ATP-binding cassette protein ABCB5, a single molecular marker, can be used to isolate dermal cell subpopulation of the skin with multipotent mesenchymal stromal cell (MSC) characteristics from its endogenous niche. The ABCB5⁺ MSCs maintain most of their stemness and mesenchymal marker during large in vitro expansion cultures as well as the capacity for clonal self-renewal and, importantly, promote healing of non-healing iron-overload wounds in a murine model, which may be exploited as a potential regenerative therapy for chronic venous leg ulcers in human patients.

### Human and Murine Dermis Harbor ABCB5⁺ Stromal Cells in the Perivascular and Interfollicular Niche

Using immunostaining of healthy human skin sections, it was demonstrated that

ABCB5⁺ cells co-stain for the carbohydrate stage-specific embryonic antigen-4 (SSEA-4), an embryonic germ and stem cell marker [10] earlier reported to be expressed on MSCs in different adult tissues, including the dermis [11, 12, 13].

Interestingly, ABCB5⁺ cells were either confined to a perivascular endogenous niche, in close association with CD31+ endothelial cells or were dispersed within the interfollicular dermis independent of hair follicles. ABCB5⁺ cells constituted 2.45%±0.61% of all dermal cells in the skin of ten different donors and of the ABCB5⁺ cells, 55.3%±23.9% were localized perivascularly, which was defined as a maximum of one additional cell in between the CD31+ endothelial cell and the ABCB5⁺ cell. Perivascular ABCB5⁺ cells were distinct from neural/glial antigen 2 (NG2) positive pericytes [14], as there was almost no co-localisation of NG2 with ABCB5 in double immunostained human skin sections. A similar distribution of ABCB5⁺ cells in their endogenous niche was found in murine skin.

Moreover, RNA seq analysis from ABCB5⁺ enriched MSCs—even when expanded in culture to high passage numbers—revealed expression of distinct stemness as well as mesenchymal marker genes. Furthermore, the expression of selected stemness markers such as SSEA-4, DPP4 (CD26), PRDM1 (BLIMP1) and POU5F1 (OCT-4) in ABCB5+ cells in human skin at protein level was confirmed by immunostaining. While the expression of lower fibroblast lineage marker α-smooth muscle actin (α-SMA) was absent in ABCB5⁺ cells of human skin. Together these results support stemness properties of ABCB5⁺ cells that are at least in part maintained in vitro and can be exploited therapeutically for the treatment of non-healing wounds.

### Human Dermal ABCB5 Cells Reveal Mesenchymal Stem Cell Properties

To assess whether selection for ABCB5 results in a cell fraction with MSC properties, dermal single cell suspensions derived from enzymatically digested skin were separated by multiple rounds of ABCB5 magnetic bead sorting. This resulted in two different cell fractions, a double ABCB5-enriched fraction containing on average 98.33%±1.12% ABCB5⁺ cells and a threefold ABCB5⁻ depleted fraction, that only contained a very low percentage of ABCB5⁺ cells as illustrated with flow cytometry dot plots for calls from donor B01 (Tables 1A-B). Both ABCB5+ and ABCB5⁻ fractions displayed a fibroblastoid, spindle-like cell morphology and expressed the characteristic minimal set of mesenchymal lineage markers CD90, CD105 and CD73, while no expression of hematopoietic stem cell and lineage markers CD34, CD14, CD20 and CD45 [15] was detected by flow cytometry. A consistent and significantly increased potential for adipogenic, osteogenic and chondrogenic lineage differentiation was observed for ABCB5⁺ cells as compared to donor-matched ABCB5-depleted cells, thereby delineating the ABCB5⁺ fractions as multipotent adult MSCs from ABCB5-human dermal fibroblasts (HDFs). This was further confirmed by the finding that ABCB5⁺ sorted cells gave rise to single cell derived colonies, whereas the ABCB5-depleted fractions did not. To assess the in vitro self-renewal capacity of dermal ABCB5⁺-derived MSCs, subclonogenic growth and tri-lineage differentiation potential of 54 clonal cultures of ABCB5⁺ sorted MSCs from six different donors were determined. Interestingly, 75.61±16.86% of clonal colonies again displayed clonogenic growth and 62.40±7.54% of all studied clones, generated from a single cell, and maintained their potential to differentiate into all three mesenchymal cell lineages. An additional 29.84±11.57% of these clones were bipotent, and 7.77±10.02% were unipotent for osteogenic differentiation. None of the clones from six donors were negative for all three lineages. When compared to the gold-standard of bone marrow derived MSCs with a tri-lineage differentiation capacity of 34% in more than 200 studied single cell clones [16], the tri-lineage differentiation capacity >70% was apparently better in ABCB5⁺ skin-derived MSCs.

In contrast to triple ABCB5-depleted cells, the ABCB5⁺ sorted cell fractions revealed distinct stem cell associated SSEA-4 [17] expression. This matches with the observed co-expression of ABCB5⁺ cells with SSEA-4 in human skin. Nuclei of ABCB5⁺ cells grown on slides stained positive for SOX2, the stem cell-associated transcription factor sex determining region Y-box 2, whereas ABCB5− cells did not. Neither ABCB5⁺ nor ABCB5⁻ dermal plastic-adherent cell fractions expressed the additionally tested cell surface markers Melan-A (melanocytic cells), CD133 (cancer stem cells), CD318 (epithelial cells) and CD271 (a neurotrophic factor found on other MSC populations).

Human ABCB5⁺-Derived MSCs Accelerate Wound Healing in Iron Overload Mice Through Triggering a Switch from M1 to M2 Macrophages

In order to address whether the here characterized dermal ABCB5⁺-derived MSCs exert anti-inflammatory effects on classically activated M1 macrophages, ABCB5⁺-derived MSCs were co-cultured with allogeneic PBMC CD14⁺ monocyte-derived macrophages that had been activated with recombinant human IFN-γ and LPS. Of note, significantly less M1 macrophage derived pro-inflammatory cytokines TNFα and IL-12/IL-23p40 were detected in supernatants when activated macrophages were co-cultured with ABCB5+-derived MSCs, as opposed to co-cultures with donor-matched ABCB5⁻ HDFs or macrophages cultured alone. Conversely, increased amounts of IL-10, a M2 macrophage derived anti-inflammatory cytokine, were found in supernatants of macrophages co-cultured with ABCB5⁺-derived MSCs as opposed to donor-matched ABCB5⁻ HDFs or macrophages cultured alone. Of note, pooled ABCB5⁺-derived MSCs from 6 different donors revealed a similar suppressive action on M1 macrophage cytokines with a concomitant up-regulation of the M2 macrophage cytokine IL-10 when compared to the single ABCB5⁺-derived MSCs. These data imply that pooled preparations of ABCB5⁺-derived MSCs would be a practically relevant option for the treatment of non-healing wounds in clinical routine.

Similar to co-cultures of human ABCB5⁺-derived MSCs with human macrophages, human ABCB5⁺-derived MSCs exert identical effects on murine macrophages in a cross-species setting, thereby confirming functional relevance for subsequent wound healing studies in a murine xenograft model.

Next, in order to specifically investigate the paracrine effects of ABCB5+-derived MSCs on suppression of M1 macrophages, which—due to their unrestrained activation—are responsible for the non-healing state of chronic human wounds, the iron overload mouse model was employed [7] with full thickness excisional wounds in a xenograft setting. The iron overload wound model faithfully recapitulates major pathogenic aspects of chronic venous leg ulcers [7]. ABCB5⁺-derived and ABCB5-depleted dermal human cells were injected into the dermis around the wound edges at day one after wounding. The persistence of injected human cells at day three after wounding was confirmed by immunostaining for the human major histocompatibility complex I constant subunit β2-microglobulin (β2M). By means of human-specific beta actin sequence PCR on genomic DNA isolated from wound sections, persistence of human-specific beta actin-signals was confirmed to a similar extent in the wounds injected with either ⁺-derived MSCs or ABCB5⁻ cells at indicated time points. Therefore, differences in the persistence between ABCB5⁺ and ABCB5⁻ cells did not confound the results.

The question whether injection of ABCB5⁺-derived MSCs accelerate wound closure in the iron overload model was addressed next. As expected, delayed wound closure was observed in iron-treated/PBS injected mice as compared to dextran-treated/PBS-injected control mice. Of note, a significantly accelerated wound closure was observed after intradermal injection of 106 ABCB5⁺-derived MSCs around 4 wounds (per mouse) compared to injection of donor-matched ABCB5-HDFs or PBS alone. Treatment with ABCB5⁺-derived MSCs fully restored the wound closure rate to that of dextran-treated/PBS-injected control mice.

Together these findings suggest beneficial effects of ABCB5⁺-derived MSCs for the cure of non-healing chronic wounds.

### Human ABCB5⁺-Derived MSCs Suppress Inflammation and Improve all Subsequent Wound Phases in Iron Overload Mice

Chronic wounds persist in the inflammatory wound phase with unrestrained M1 macrophage activation, and fail to progress through the normal phases of wound healing. It was here studied whether injection of ABCB5⁺-derived MSCs may suppress the unrestrained M1 macrophage-dependent inflammation, and allow the wounds to follow the normal sequence of different wound phases. Employing double immunostaining, ABCB5⁺-derived MSCs were found in close association to endogenous murine macrophages when injected in iron overload wounds, implying that a paracrine effect of ABCB5⁺-derived MSCs on macrophages is possible in wound tissue. In a first attempt to explore a paracrine impact of ABCB5⁺-derived MSCs on macrophage dominated inflammation in iron overload wounds, whole wound cytokine profiles were studied by ELISA on protein lysates. Notably, at day five after wounding, wound tissue protein levels of the inflammatory cytokine TNFα were dampened, whereas anti-inflammatory IL-10 was increased in iron overload wounds injected with ABCB5⁺-derived MSCs but not with ABCB5-HDF controls. Furthermore, the inflammatory cytokine IL-1β, that is typically up-regulated in human CVU and in iron-overload murine model, was significantly suppressed upon treatment with ABCB5⁺-derived MSCs.

Faster re-epithelialization was also observed with a fully restored K14+ epithelial cell layer covering the entire wound bed, a key feature of successful skin repair, in day seven iron overload wounds when injected with ABCB5⁺-derived MSCs as opposed to ABCB5⁻ HDF injected wounds. A significant improvement of neo-vascularization was observed as confirmed by increased number and area of CD31⁺ vessel sprouts within the wound bed at day seven. In addition, injection of ABCB5⁺-derived MSCs in wound edges of iron overload mice markedly improved the tissue remodeling with increased maturation of collagen fibers, reduced granulation tissue depth and improved organization of collagen fibers in more densely basket-woven fibrillary structure. Of note, iron overload wounds injected with ABCB5⁺-derived MSCs depicted a significantly higher tensile strength of the scar tissue, a strong indication for improved quality of the restoration tissue, as compared to less tensile strength in scar tissue of ABCB5⁻ HDF or PBS treated iron overload wounds. These data show that ABCB5⁺-derived MSCs positively impact on several wound healing phases, and not only accelerate tissue repair, but importantly, lead to a scar-reduced, quality-improved restoration tissue.

### ABCB5⁺-Derived MSCs Suppress Macrophage Dominated Inflammation Via Adaptive Secretion of IL-IRA

Given the abundance of IL-1β and its inflammation amplifying effector TNFα [7] in chronic wounds as opposed to transiently induced low IL-1β concentrations in acute wounds, the question as to whether human dermal ABCB5⁺-derived MSCs are able to produce the natural antagonist of IL-1 signaling, IL-IRA, was addressed. It was found that unstimulated ABCB5⁺-derived MSCs in culture did not readily produce IL-1RA as assessed by a specific ELISA. However, in contrast to donor-matched ABCB5⁻ HDFs, ABCB5⁺-derived MSCs released high IL-IRA levels when stimulated with IFN-γ/LPS. Of note, the IL-IRA concentration was even higher in co-cultures of ABCB5+-derived MSCs with IFN-γ/LPS activated M1 macrophages. Six hours after injection, specific IL-1RA expression was observed in ABCB5⁺-derived MSCs at the wound site of iron overload mice as shown by double immunostaining with distinctly co-localized human-specific (32M and IL-1RA. Employing Western blot analysis, high IL-1RA expression was confirmed in pooled day three wound lysates prepared from iron overload ABCB5⁺-derived MSCs injected wounds as compared to no IL-1RA expression in ABCB5⁻ HDFs or in PBS injected control wound lysates. Of note, and previously unreported, IL-1RA expression was also observed in endogenous murine ABCB5⁺ MSCs in iron overload model wound healing, while in healthy skin, neither murine nor human endogenous ABCB5⁺-derived MSCs were found to express IL-1RA. These data imply an adaptive production of IL-1RA by dermal ABCB5+ MSCs in response to the inflammatory environment of iron overload wounds. A small fraction of murine macrophages, but not neutrophils, release IL-1RA in iron overload chronic wounds. The therapeutic impact of IL-1RA released from ABCB5⁺-derived MSCs on acceleration of healing of iron overload wounds is, however, significantly more important, as IL-1RA-silenced MSCs, when injected into iron overload wounds, cannot restore delayed wound healing. It was next explored whether IL-1RA released by ABCB5⁺-derived MSCs are responsible for the suppression of M1 macrophage derived TNFα in vitro and in vivo. ABCB5⁺-derived MSCs were assessed for TNFα release in wounds supernatants of iron overload mice injected with either IL-IRA silenced or competent ABCB5⁺-derived MSCs. Notably, silencing of IL-IRA in ABCB5⁺-derived MSCs at least partially abrogated TNFα suppression ico-cultures with either human or murine macrophages. As expected, scrambled control siRNA transfected IL-1RA competent control ABCB5⁺-derived MSCs revealed their full suppressive effect on TNFα release from activated macrophages in vitro. Strikingly, intradermal injection of IL-1RA silenced ABCB5⁺-derived MSCs into wound edges of iron overload mice resulted in a complete loss of accelerated wound closure. By contrast, scrambled siRNA transfected IL-1RA competent ABCB5+ MSCs maintained their capacity to accelerate wound healing at the indicated time points in vivo. The loss of the capacity of IL-1RA silenced ABCB5+ MSCs to accelerate healing in iron overload wounds was associated with a reversal of TNFα and IL-1β suppression and IL-10 up-regulation. These data indicate that IL-1RA adaptively released from ABCB5+ MSCs upon stimulation at the wound site not only suppresses IL-1 signaling, but also the downstream effector TNFα and, importantly, even induces anti-inflammatory IL-10. The notion that IL-1RA released from ABCB5⁺-derived MSCs at the wound site suppressed unrestrained M1 activation with improved wound healing is further supported by the finding that intradermal injection of recombinant human IL-1RA around iron overload wounds also accelerated wound closure. By contrast, injection of recombinant IL-1RA into acute wounds did not accelerate healing. TSG-6 was also found to be expressed in ABCB5 human MSCs in iron-overload wounds. However, when injecting recombinant TSG-6 into iron overload wounds, no improvement of wound closure occurred. The results imply that IL-1RA, indeed, plays a central role in iron overload wounds, while recombinant human TSG-6 alone is not sufficient to accelerate healing in the iron overload situation. This implies that different wound types reveal distinct requirements for therapeutic acceleration of their healing.

### ABCB5⁺-Derived MSCs Break M1 Macrophage Persistence in Wounds of Iron Overload Mice

To further sustain the hypothesis that wound treatment with ABCB5⁺-derived MSC would IL-1RA-dependently break the prolonged persistence of M1 macrophages in wounds of the iron overload mice, a series of double immunostainings of day five wound sections were performed. In fact, TNFα expressing F4/80+ macrophages were virtually absent in iron overload wounds injected with ABCB5⁺-derived MSCs. In stark contrast, many TNFα+F4/80+ double positive macrophages persisted in wound margins upon injection of IL-1RA silenced ABCB5+-derived MSCs similar to dextran pre-treated acute healing control mice. These data indicate that ABCB5+-derived MSCs IL-1RA-dependently suppress wound macrophage released TNFα production in vivo. Interestingly, CD206+F4/80+ wound healing promoting M2 macrophages appeared to be IL-1RA-dependently enriched in ABCB5+-derived MSCs injected wounds at day five post wounding. In fact, immune-phenotyping of single cell preparations of day five wounds injected with ABCB5⁺-derived MSCs quantitatively confirmed an IL-1RA-dependent switch of inflammatory M1 towards wound healing promoting M2 macrophages as defined by distinct sets of surface markers. Thus, M1 activation markers, including cytokines (TNFα, IL-12/IL-23p40) and the inducible nitric oxide synthase (NOS2), were down-regulated and M2 activation markers like the mannose receptor CD206, the β-glycan Dectin-1 and arginase-1 (ARG1), were upregulated in F4/80⁺ wound macrophages after ABCB5⁺-derived MSCs injection. This M1 to M2 shift was maintained in scrambled siRNA transfected ABCB5⁺-derived MSCs, while it was almost completely abrogated following injection with IL-1RA siRNA transfected ABCB5⁺-derived MSCs. In aggregate, these results uncover a causal role for IL-1RA to abrogate persistence of M1 macrophage in chronic wounds secreted by ABCB5⁺-derived MSCs.

### The ABCB5⁺-Derived MSCs-Dependent M1 to M2 Macrophage Shift is Conserved in Humanized NSG Mice

NSG mice, humanized with PBMC, represent a highly suitable preclinical model to investigate effects of therapeutic interventions on human hematopoietic lineage derived cells in vivo [18]. This model was employed here to validate the effect of ABCB5⁺-derived MSC injection on the M1/M2 wound macrophage phenotype of human origin in NSG iron overload mice. For this purpose, full thickness wounds were inflicted on PBMCs humanized NSG mice with subsequent intradermal injection of either human allogeneic ABCB5⁺-derived MSCs, donor-matched ABCB5-HDFs, or with PBS alone into the wound edges. In line with the above findings, accelerated closure of full thickness wounds upon injection with ABCB5⁺-derived MSCs was observed compared to PBS and ABCB5-HDF injection of wounds in PBMC-humanized NSG mice. Co-immunostaining of day five wounds with human specific anti-CD68 and either anti-CD206 or anti-TNFα showed a higher number of CD68+CD206+ human M2 macrophages in the wound beds of ABCB5⁺-derived MSC-injected compared to PBS-injected wounds. Of note, the number of CD68+ TNFα+ pro-inflammatory macrophages was decreased in ABCB5⁺-derived MSCs compared to PBS injected wounds. Single cell suspensions derived from day 5 wound tissue were analyzed by multi-color flow cytometry in order to confirm the numbers of human CD68+M1 and M2 macrophages at the wound site. The ratios of human M2 to M1 macrophage marker expressing CD68+ human macrophages were increased in wound tissue treated with ABCB5⁺-derived MSCs compared to PBS for both the ratio of Dectin-1/IL-12p40 and CD206/TNFα expressing cells. These data indicate that the beneficial anti-inflammatory effect of IL-1RA released from ABCB5⁺-derived MSCs on human wound macrophages was conserved in humanized NOD-scid IL2rγ^{null} mice.

### Discussion

A newly defined dermal cell subpopulation of the skin with MSC characteristics can be successfully isolated from its endogenous niche by a single marker, the P-glycoprotein ABCB5, and developed to a high purity and homogeneity. The isolated ABCB5⁺ MSC subpopulation reliably maintains the capacity of clonal self-renewal and clonal tri-lineage differentiation. Injection of the newly synthesized ABCB5⁺ lineage-derived MSCs around wounds—via paracrine IL-1RA release—switch pro-inflammatory M1 macrophages with unrestrained activation to anti-inflammatory wound repair promoting M2 macrophages in chronic iron overload wounds and, in consequence, accelerate impaired wound healing in vivo. This constitutes a major breakthrough at the forefront of MSC-based therapies in translational medicine which—due to the lack of an appropriate selection marker—a problem with existing therapeutic applications of less characterized MSC populations with inconsistent efficacy and potency [19].

The development of a highly effective synthetic stem cell populations from skin MSCs depends on the exclusive expression of ABCB5 on MSCs, but not on other cells in the skin. Employing a global transcriptomic approach, the existence of dermal ABCB5⁺ cells with a MSC characteristic cell surface expression profile was developed, and co-expression with additional pluripotency and stem cell markers is herein reported. Evidence is also provided from RNA seq analysis that synthetic ABCB5⁺ cells, even when expanded in culture to high passage numbers, maintain at least in part their stemness, MSC and mesenchymal marker expression of endogenous ABCB5⁺ cells in the skin. The data disclosed herein suggest that the synthetic ABCB5⁺-derived MSCs may share some expression features of scar-reducing upper lineage fibroblasts.

An impressive rescue of impaired wound healing in virtually all studied phases of iron overload wounds, indeed, depends on enhanced IL-1RA release from injected ABCB5⁺-derived MSCs, which actively shifted prevailing unfavorable M1 macrophages to wound healing promoting M2 macrophages. This finding is of particular clinical interest given the shared pathogenic role of unrestrained activation of pro-inflammatory M1 macrophages causing impaired wound healing in difficult-to-treat chronic wounds in humans [7, 8, 22]. Several lines of evidence supported this finding.

First, injection of synthetic ABCB5+, but not of ABCB5-depleted dermal cells resulted in enhanced repair of impaired wound healing in iron overload mice. Within the wound bed of iron overload mice, M2 macrophages were more abundant after ABCB5⁺-derived MSCs injection in contrast to persisting high numbers of over-activated M1 macrophages as found in iron overload wounds after injection of either PBS or ABCB5-depleted dermal cell fractions. Second, the occurrence of M2 macrophages in wound beds of ABCB5⁺-derived MSC-injected iron overload wounds was associated with an increase of anti-inflammatory IL-10, a typical M2 cytokine which suppresses inflammation. At the same time, a decrease of the classical M1 macrophage cytokines TNFα, IL-1, IL-12 and IL-23, only important during early wound healing phases in recruiting and activating microbiocidal M1 macrophages [23] was observed. Third, the previous data showed that under M1 macrophage depleting conditions iron overload wounds depicted a fully restored switch to M2 macrophages with improved wound healing similar to non-iron overload wounds [7].

As to the question why IL-1RA—apart from IL-1 β can significantly reduce also TNFα, the following scenario is most likely. Both TNFα and to a higher extent IL-1β concentrations are increased in the iron overload murine wound model, both cytokines driving activation of inflammatory cells, particularly macrophages. Both IL-1β and TNFα can activate NFκB [24, 25], which itself transactivates target genes such as IL-1β, IL-6 and TNFα among other pro-inflammatory cyto- and chemokines. In consequence, if IL-IRA neutralizes the high amounts of IL-1β, it is expected that the vicious cycle of NFκB activation is significantly reduced with overall less activation and expression of target genes such as IL-1β and TNFα. As IL-1β predominantly enforces its effect via IL-6 induction [24, 26], IL-IRA most likely would impact on overall IL-6 concentrations, and consequently on NFκB activation and downstream target genes. Certainly, other driver cytokines beyond IL-1β and TNFα cannot be excluded. What can be concluded from the data is that IL-IRA released from synthetic ABCB5+ MSCs does play a causal role in rebalancing the hostile microenvironment of chronic iron overload wounds.

It is likely that the inflammasome, a multiprotein complex, is responsible for the enhanced release of IL-1β in the iron overload wound model. In fact, both iron as well as constituents of bacteria contaminating chronic wounds promote inflammasome overactivation [27, 28]. The role of the inflammasome in acute and chronic tissue damage is complex and far from being fully understood. Transient activation of the inflammasome during physiological wound healing is a prerequisite to coordinate the inflammatory response in defense against microbial invasion and to effectively remove tissue debris [28]. The inflammasome-dependent maturation of IL-1β occurs via cleavage of the pro-peptide through caspase 1 and is necessary to recruit and activate neutrophils and macrophages to the site of injury. Inhibition of this inflammasome-dependent maturation step of IL-1β in mice deficient for caspase 1 revealed delayed wound healing [29]. Unrestrained activation of IL-1β in mice deficient of the IL-1 receptor antagonist IL-IRA resulted in a fibrotic response of lung tissue in a model of Chlamydia pneumoniae infection [30]. Similar to the present data, persistent inflammasome-dependent activation of IL-1β in diabetic mice also correlates with delayed wound healing of skin wounds [31] which can be almost completely restored to normal healing by suppression of the inflammasome [32]. The findings show that balanced inflammasome activation is crucial for coordinated tissue repair, and if this balance is disrupted wound healing will be impaired.

Descriptive evidence that MSCs dampen single aspects of macrophage activation in vitro [33, 34, 35, 36, 37] and even in acute wound models have been reported [33, 36, 37, 38]. However, a thorough characterization of the switch from M1 to M2 macrophages or the responsible paracrine mechanism is lacking. Therefore, the present approach highlights the usefulness of a more complete assessment of the paracrine effects of ABCB5⁺-derived MSCs on healing of chronic wounds and helped to identify IL-1RA as the key effector molecule responsible for a rigorous switch from pro-inflammatory, detrimental M1 macrophages to anti-inflammatory M2 macrophages.

The data on the paracrine effect of IL-1RA released from ABCB5⁺-derived MSCs are in line with previous findings [39]. In this regard, IL-1RA knock-out mice displayed delayed wound healing of acute wounds [39]. Furthermore, improved healing was reported in mice with a targeted deletion of the IL-1 receptor (IL-1R) or after treatment with recombinant IL-1RA of acute wounds of wild-type mice [40] and of diabetic mice [8]. IL-1RA secretion from less well characterized MSCs has been described to be beneficial in a variety of pathological conditions in preclinical studies [41]. The understanding that the shift from the unrestrained pro-inflammatory M1 to the anti-inflammatory M2 macrophages is due to the beneficial IL-1RA effects reliably controlling macrophage dominated tissue inflammation is herein distinctly advanced.

In line with the concept and data, there is clear evidence from the literature [42] that human IL-1RA can efficiently bind to murine cells with a high affinity and thereby inhibit murine IL-1β binding and signaling. In this regard, human IL-1RA has earlier been shown to bind to the type I IL-1 receptor on murine cells with an affinity of 150 pM, equal to the binding of human IL-1α and IL-1β.

The present findings cannot exclude that, in addition to IL1RA, other mechanisms may contribute to counteract tissue damage due to unrestrained M1 macrophage activation. In fact, several investigators including ourselves have earlier shown that MSCs dampen inflammation and, in consequence, reduce scar formation in tissue repair via the release of tumor necrosis factor-inducible gene 6 protein (TSG-6) [36, 43]. By contrast to accelerated healing of full thickness wounds following TSG-6 release from MSCs injected at the wound site [36], though TSG-6 was expressed at the wound site of iron overload wounds, TSG-6 apparently does not play a major role in accelerating healing of iron overload wounds. In fact, injection of recombinant TSG-6 at concentrations which enhance acute wound healing, does not enhance healing of iron overload wounds. Differences in the microenvironment will be sensed by injected MSCs which, in consequence, may raise different adaptive responses in terms of the anti-inflammatory factors released.

Apart from IL-1RA, other factors may contribute to the accelerated healing. In this regard, MSCs have been reported to suppress oxidative damage during sepsis via PGE2-dependent reprogramming of macrophages to increase the release of anti-inflammatory IL-10 [44]. In addition, by enhanced IL-6 and TGF-β release, MSCs inhibit neutrophil recruitment by cytokine activated endothelial cells [45].

A minor limitation of the murine wound model employed is the modest delay in wound closure compared to non-healing CVU in patients. Nevertheless, this model mimics the iron-induced unrestrained activation of wound M1 macrophages with prolonged inflammation and tissue break down and, hence, represents a well-suited model to study the effect of treatment strategies on these specific pathophysiological traits [7].

In aggregate, the findings have substantial clinical impact for the planned implementation into clinical routine. Here, the adaptive release of a key factor efficiently dampening unrestrained M1 macrophage dominated inflammation underlying dysregulated tissue repair in iron overload chronic wounds was first uncovered. Second, the employment of a single marker strategy allows the enrichment of an easily accessible homogeneous ABCB5⁺-derived MSC population from human skin with GMP grade quality, ready to use for transition into clinics. Third, an in vitro assay predictive for the successful action of the employed MSC preparations in a chronic murine wound model was developed. ABCB5⁺-derived MSC preparations from different donors, alone or pooled, successfully suppressed the release of M1 macrophage cytokines and this suppressive effect correlated well with the improvement of healing when the corresponding ABCB5⁺-derived MSCs were injected into iron overload wounds.

Thus, the above data reveal enhanced efficacy and potency of the newly described dermal ABCB5⁺-derived MSCs, which hold substantial promise for the successful clinical therapy of non-healing wounds. In fact, a clinical phase II study has recently been initiated (EudraCT number: 2015-000399-81) with promising results of the first studied patients.

### Materials and Methods

### Study Design

The purpose of this study was to determine whether human dermal ABCB5+ cells are MSCs and have beneficial effects on chronic wound healing in cellular therapeutic applications. In vitro, ABCB5+ MSCs and donor-matched ABCB5-HDFs from at least six different donors (Table 1: B02-B07) were tested for MSC-characteristic tri-lineage differentiation, surface marker expression, clonogenic growth, self-renewal, and anti-inflammatory effects on activated macrophages by quantitative measures. In vivo, improvement on wound healing by anti-inflammatory mechanisms was assessed in the mouse iron overload full-thickness excisional wound model for chronic venous ulcers, characterized by delayed wound closure, prolonged inflammation and M1 activated macrophage abundance [7]. For these animal studies, sample sizes were estimated based on differences in wound closure from the previous study identifying delayed wound healing in genetically modified mice [46] in order to reach a significance level of 5% and a statistical power of 80% by the Welch's test, with the inclusion of one additional animal (four wounds) to protect against deviations from the Gaussian distribution. Key animal experiments with ABCB5⁺-derived MSCs and donor-matched ABCB5-HDF injection were performed three times with cells from three different donors (Table 1: B01, B13, B14). Repetition experiments for sample collection were performed with human dermal cells either from the donor with internal number B01 for which cell preparation purities and wound closure data are shown here, or with a phenotypically and functionally verified pooled sample of cells from six different donors (Table 1). This pooled dermal ABCB5⁺-derived MSC preparation was also used for Il-1RA knock-down and humanized NSG mouse wound closure experiments. The amount of independent biological samples analyzed in each quantitative assay. Microscopic images are representative for six wound samples per treatment group. Biological samples for analysis of xenografted ABCB5⁺-derived MSC persistence by human-specific beta actin qPCR on wound sections and ELISA quantification of wound cytokine titers are each pooled from two independent wounds and for hIL-1RA Western Blot and wound macrophage flow cytometry from four independent wounds.

### Human Skin Samples

Skin biopsies used for the isolation of ABCB5+ and ABCB⁻ cell fractions in this study measured 1 cm2 and were either taken from young healthy volunteers at the University Clinic of Dermatology and Allergic Diseases in Ulm, the University Clinic of Gynecology (skin from healthy females undergoing reduction mammoplasty) (Donors B02-B07) after approval by the ethical committee at Ulm University or directly derived from clients of Ticeba GmbH (Heidelberg, Germany) (Donors B01, B08-B14) according to the Declaration of Helsinki principles after informed written consent was obtained. Localization was chosen to avoid isolation of cells from sun-exposed areas of the skin. The variation in localization (gluteal region, inner upper arm or behind left ear) depended on surgical standards and donor preference. All biopsies were histologically assessed for any pathology. Only biopsies without pathology were employed for immunostaining or for isolation of ABCB5⁺ and ABCB− cell fractions. None of the biopsies taken failed to yield ABCB5⁺ cells. Anonymized donor-data can be found in Table 1. Expansion of plastic-adherent dermal cells and ABCB5-based separation modified from Frank et al. [47] was performed as indicated (see materials and methods for details). Cell viability was assessed prior to in vitro experiments, and no difference was found between both in the ABCB5⁺ and ABCB5⁻ population (>90%). Also, when harvesting ABCB5+ MSCs and the ABCB5-cell fraction by Accutase for the injection into wounds, viability is routinely checked by trypan blue exclusion and is consistently very high (>90%) both in the ABCB5⁺ and ABCB5⁻ population.

Before application in in vivo wound healing experiments, ABCB5⁺ cell preparations were tested for their M1 macrophage suppressing function in a co-culture with IFN-γ/LPS activated murine bone marrow-derived macrophages and the release of TNFα was assessed by a mouse-specific TNFα ELISA (R&D Systems).

### Differentiation and Clonogenic Growth Assays

In vitro adipogenic, osteogenic and chondrogenic differentiation capacity was examined using commercial differentiation media (Lonza); TGF-β3 (CellSystems) and procedures according to manufacturer's descriptions. For adipogenic differentiation, lipid droplet accumulation was verified by staining with Oil Red 0 (Sigma-Aldrich) and quantified by dye extraction as described previously [48]. Mineralization of the extracellular matrix of osteoblasts was verified by Alizarin Red S staining (Sigma-Aldrich), and quantified by subsequent dye extraction as described [49]. To visualize chondrogenic differentiation, 3D-micromass cultures were immunostained for Aggrecan (R&D Systems, AF1220) according to standard procedures (see section “Immunofluorescence staining”). For quantification of chondrogenesis, cartilage-specific sulphated proteoglycans and glycosaminoglycans formed in the micromasses were measured using the Blyscan Glycosaminoglycan Assay kit (Biocolor) according to the manufacturer's instructions. For assessment of clonogenic growth, ABCB5⁺ dermal MSCs and donor-matched ABCB5⁻ HDFs were seeded at a density of 200 cells per 100 mm culture dish. After 14 days, colonies were stained with 0.5% crystal violet (Sigma-Aldrich) and colonies ≥25 cells were counted on three to five parallel dishes per sample. For clonal expansion assays, ABCB5⁺-derived MSCs were seeded at 100 cells per 100 mm culture dish. After 14 days, 12 colonies separated from neighboring colonies by at least one microscopic field were picked and expanded. Well growing clonal cultures were elected for secondary tri-lineage differentiation and clonogenic growth assays.

### Human and Mouse Macrophage Co-Cultures

Mouse bone marrow-derived macrophages were isolated from femurs and matured for six days with macrophage colony-stimulating factor (M-CSF) containing L929 cell supernatant supplementation as described [46]. Human macrophages were matured under presence of 20 ng/ml recombinant human M-CSF (Miltenyi Biotec) for eight days from PBMC-derived monocytes sorted for CD14 expression by positive magnetic bead selection (Miltenyi Biotec) with purity >95%. Fresh buffy coats for PBMC isolation by gradient centrifugation (PAA) were obtained from the German Red Cross. For co-culture experiments, ABCB5⁺-derived MSCs or donor-matched ABCB5-HDFs were plated to adhere at 2×104 cells/well in 24-well plates in 0.5 ml DMEM with 10% high quality fetal bovine serum, 100U/ml penicillin/streptomycin and 2 mM L-glutamine. After 24h macrophages were seeded on top at 1×105 cells/well in 0.5 ml, resulting in a 1:5 cell ratio unless indicated differently. Co-cultures were incubated with 50 U ml−1 recombinant mouse or human IFN-γ (R&D

Systems) for 24h and then stimulated with 20 ng ml−1 LPS (Sigma-Aldrich) and 50 U ml−1 IFN-γ for another 24h period before supernatants were harvested and analyzed by ELISA (R&D Systems).

### Mice and Wound Healing Models

Both female C57BL/6N (Charles River, strain 027) and female or male NOD.Cg-Prkdcscid Il2rgtm1Wj1/SzJ (Jax strain 005557) mice were 10-12 weeks at the start of experiments and held under specific pathogen-free conditions in individually vented cages at the animal facility of the University of Ulm. Experiments were performed in compliance with the German law for welfare of laboratory animals and approved by the Baden-Württemberg governmental review board.

The C57BL/6 mouse model relevant for CVU physiopathology was performed as described previously [7]. For cellular treatment with human dermal ABCB5⁺-derived MSCs or corresponding ABCB5-HDFs, 1×106 cells suspended in PBS per mouse were injected into thdermis at three 50 μl injection points around each wound edge.

For the assessment of wound closure, NSG mice were humanized with 2×107 human PBMC in 200 μl PBS by tail-vein injection as previously described [18] eight days before wounding. At day one post-wounding, mice were randomly assigned to treatment groups receiving intradermal injection of either a six-donor pool ABCB5+ MSC preparation (Table 1: B01+B08+B09+B10+B11+B12), donor-matched ABCB5⁻ HDFs or PBS alone. For the assessment of macrophage phenotype shift, NSG mice were humanized one day prior to wounding. At day one post-wounding, random groups were treated with either ABCB5⁺ MSCs from donor B01 or PBS as described above. At day five after wounding, two independent wound halves of each mouse were processed for immunofluorescence staining, the others were pooled for flow cytometry.

siRNA-Mediated Knock-Down of IL-IRA Expression in ABCB5⁺-Derived MSCs

ABCB5⁺-derived MSCs were transiently transfected with 20 nM of either a combination of four siRNAs specific for human IL-1RA or with scrambled control-A siRNA with accompanying transfection medium at the minimum recommended concentration (all products from Santa Cruz Biotechnologies) according to manufacturer's instructions. Successful knock-down was tested at the protein level upon in vitro inflammatory stimulation with IFN-γ/LPS activated mouse bone marrow-derived macrophages and culture supernatant medium ELISA for human IL-1RA (R&D Systems) before use in in vivo experiments and was typically at ˜80%.

### Histology and Immunofluorescence Staining

Human skin tissue samples were embedded in O.C.T. compound (TissueTek), frozen at −80° C. processed to 5 μm sections and fixed in acetone. Mouse wounds were fixed overnight with 4% PFA, cut through the middle, paraffin embedded and only the first series of 5 μm sections were used to avoid the wound edges. Adherent cells were cultured on glass coverslips, fixed with 4% PFA and permeabilized with 0.5% TritonX-100 in PBS. Sections or slides were incubated with primary antibodies listed in supplemental materials (Table 3) that were diluted as per manufacturers recommendations in antibody diluent (DAKO) at 4° C. overnight. Mouse anti-ABCB5 was used at a concentration of 14 μg ml-1 and incubation of 40 minutes at 37° C. for staining of cryosections and 4 μg ml-1 at 4° C. overnight for staining of adherent cells. After washing with PBS, sections or slides were incubated with either AlexaFluor488 or AlexaFluor555-conjugated corresponding secondary antibodies (all from Invitrogen). Nuclei were counterstained with DAPI before mounting in fluorescent mounting medium (DAKO). The background staining was controlled by appropriate isotype matched control antibodies. The specificity of the anti-ABCB5 staining was assessed by a peptide competition assay, pre-incubating the antibody with a 200 fold molar excess of peptide of the epitope amino acid sequence [47], RFGAYLIQAGRMTPEG (SEQ ID NO:1), GeneCrust) prior to immunofluorescence staining, showing a loss of the fluorescent signal.

Masson trichrome (Sigma-Aldrich) and picrosirius red (Polysciences) stainings were performed as per manufacturer's instructions on paraffin sections and picrosirius red stained slides were analyzed with circularly polarized light. Images were captured with an Axiolmager.M1 microscope, AxioCam MRc camera and AxioVision software (Carl Zeiss).

Human-Specific Beta Actin Sequence Specific qPCR

Quantification of injected human ABCB5⁺-derived MSCs and ABCB5-HDFs within the mouse wound sections was performed by human-specific beta actin sequence PCR. Briefly, the genomic DNA has been isolated from PFA-fixed paraffin-embedded wound sections employing QIAamp DNA FFPE tissue kit (56404, Qiagen) followed by PCR with human specific beta actin primers (Forward primer: CACCACCGCCGAGACCGC (SEQ ID NO: 2) and Reverse primer: GCTGGCCGGGCTTACCTG (SEQ ID NO: 3)). Then densitometry analyses was performed to quantify the density of PCR product separated on the gel images and normalized with mouse specific beta actin sequence PCR product. The PCR of mouse beta actin was performed with mouse specific beta actin primers (Forward primer: CCTTCCTTCTTGGGTAAGTTGTAGC (SEQ ID NO: 4) and Reverse primer: CCATACCTAAGAGAAGAGTGACAGAAATC (SEQ ID NO: 5)).

### ELISAs and Western Blot

Frozen minced wound tissue samples were dissolved in RIM buffer (Sigma) supplemented with protease-inhibitor cocktail (Roche) and the phosphatase inhibitors Na3VO4 (2 mM) and NaF (10 mM) in Lysing D columns (MP Biomedicals) subjected to three rounds of 20s cooled vibrational force. Protein yield was measured by Bradford assay and spectrophotometric analysis against a BSA-standard dilution. All ELISA assays were performed with DuoSet kits (R&D Systems) following manufacturer's instructions. Western Blot analysis for IL-1RA was performed as earlier published [50]. A rabbit anti-IL-1RA IgG1 antibody (Abcam #ab124962) which detects human and murine IL-1RA at a dilution of 1:1000 and a secondary HRP-coupled anti-rabbit IgG (H+L) antibody (Dianova) at a dilution of 1:10,000 was used. Equal loading was verified by actin. Chemiluminescence was detected after addition of TMB substrate (BD OptEIA) with a Vilber Fusion Fx7 (Vilber Lourmat).

### Flow Cytometry

Flow cytometry for ABCB5 was performed using anti-ABCB5 mouse IgG1 (clone 3C2-1D12; [47]) and secondary AlexaFluor647-conjugated donkey anti-Mouse IgG (H+L) (Fisher Scientific). Multi-color labelling of cells for the MSC-marker panel CD90, CD73 and CD105 as well as for CD34, CD14, CD20 and CD45 was performed with the human MSC phenotyping kit (Miltenyi Biotec) following the manufacturer's instructions. Anti-human SSEA4-PE, CD271-FITC, CD133, CD318 and Melan-A antibodies (Table 3) were incubated with the cells for 45 minutes at 4° C. at concentrations recommended by the manufacturer. For the detection of CD133, CD318 and Melan-A, cells washed with FACS-buffer (1% BSA in PBS) were subsequently incubated with fluorochrome-conjugated secondary antibodies for 45 minutes at 4° C. Dead cells were excluded by co-staining with SYTOX Blue (Invitrogen). Isotype-matched control antibodies were used for setting of gates.

For wound macrophage isolation, mouse wounds were digested as previously described [33, 36]. Briefly, minced tissues were incubated with 1.5 mg/ml collagenase I and 1.5 mg/ml hyaluronidase I (Sigma-Aldrich) in HEPES-buffered saline for 1 h at 37° C. Single cell preparations were filtered and incubated for 15 minutes with FcR blocking (MACS) before staining with antibodies listed in supplemental materials (Table 3). Additional intracellular stainings were performed after fixation and permeabilization using a commercial kit (BD) according to the manufacturer's protocol. Blank and single stained samples were used for PMT and compensation settings. For wound macrophages, singlet F4/80+ mouse macrophages in C57BL/6N samples and singlet CD68+ human macrophages in humanized NSG mouse samples were gated for subsequent M1 and M2 marker expression analysis based on relative fluorescence units (RFU=geomean fluorescence intensity relative to isotype control sample) or % positive events within the macrophage population. Hereto, positivity thresholds were set against the relevant fluorescence-conjugated isotype controls and macrophage gating marker stained control samples. Flow cytometry was performed on FACSCanto II, FACSAria Fusion or Accuri flow cytometers (BD Biosciences) and the data thereafter analyzed using FlowJo analysis software (TreeStar Inc.).

### Comprehensive Transcriptome Profiling and Quantitative PCR

To prepare the total RNA-Seq library, 500 ng of total RNA was used as input. 500 ng of total RNA first was used to deplete the rRNA using a commercially available kit (Low Input Ribominus Eukaryotic System v2, Thermo) as described in the manual with slight modifications. In brief, after the rRNA was depleted using RiboMinus™ Eukaryote Probe Mix, the supernatant containing rRNA depleted RNA was collected and incubated with 3×Agencourt RNAClean XP beads for 20 min on ice, followed removal of supernatant and washing of RNAClean XP beads two times with 80% ethanol and finally the rRNA depleted RNA was eluted from the beads in 10 μl of nuclease free water. This rRNA depleted RNA was used to prepare RNASeq library for Illumina platform using NEBNext Ultra II Directional RNA library prep kit (NEB) with some modifications. The quality control of the RNASeq libraries were performed by Agilent Bioanalyzer and concentration of the libraries were measured in qubit using dsDNA HS assay kit (Thermo). The libraries were sequenced in 11lumina NextSeq 500 system for 75 cycles (1×75 single end reads) of sequencing and 2 index reads of 8 cycles each using NextSeq 500/550 v2 Kits (Microsynth AG, Switzerland). The demultiplex raw reads (fastq) were used for gene expression analyses as described earlier [51]. In brief, the fastq/span>files were used to align to human genome reference (GRCh38) using Hisat2, followed by transcripts assembly, abundances estimation and differential expression were performed by cufflinks and cuffdiff, respectively. The visualization of RNASeq data analyses were performed by R packages, cummeRbund, gplots, ggplot2 using customized scripts.

### Data Availability

The RNASeq data were uploaded in GEO with accession number GEO GSE125829. The 2906 base pair ABCB5 cDNA sequence can be found at NCBI GenBank under accession number AY234788.

### Statistical Analysis

Statistical analysis of in vitro and in vivo differences in independent quantitative measures between each two treatment groups was performed using two-sided unpaired Student's t-tests with Welch correction to protect against heteroscedastic data sets. In vitro comparisons for ABCB5⁺ and donor-matched ABCB5-cell fractions were analyzed by a paired t-test. On rare occasions, outliers detected by visual inspection of the data were excluded from the analysis after post hoc verification by the Grubbs' test at α=5%. Statistical data analysis was done using GraphPad Prism 6 software (Software for Science). Graphs show mean and error bars represent the standard deviation unless indicated otherwise and stars represent significance levels: ns=not significant; *p<0.05; **p<0.01; ***p<0.001.

### Materials and Methods

### Expansion and Isolation of ABCB5+ and ABCB5⁻ Dermal Cell Fractions

Plastic adherent dermal cells were expanded at the maximum for 16 passages equaling a cumulative population doubling of 25 and separated into ABCB5+ and ABCB5⁻ fractions by respective two and three consecutive rounds of magnetic bead sorting with mouse anti-human ABCB5 IgG1 antibody (clone UG3C2-2D12; (51)). More than 90% sort purity is one of the release criteria of GMP-grade dermal ABCB5⁺ cells (Table 2). By flow cytometry, average purity of ABCB5⁺ cells was 98.33%±1.12% (n =243). For experiments, sorted cells were either cryopreserved or cultured up to a maximum of 72 hours. Purity at this time-point was typically >70%. ABCB5⁺ dermal MSCs were cultured in Ham's F10 supplemented with 15% heat-inactivated high quality fetal bovine serum, 6 mM HEPES, 2.8 μg/ml hydrocortisone, 100U/ml penicillin/streptomycin, 2 mM L-glutamine, 10 μg/ml insulin, 0.2 mg/ml glucose, 6.16 ng/ml PMA (Sigma-Aldrich) and 0.6 ng/ml recombinant human basic fibroblast growth factor (Prospecbio), at 37° C. and 3% CO2. Versene (Gibco) was used to detach ABCB5⁺ dermal cells from the culture plastic. ABCB5-HDFs were maintained in DMEM with 10% high quality fetal bovine serum, 100U/ml penicillin/streptomycin and 2 mM L-glutamine (Biochrom), at 37° C. and 5% CO2.

### The C57BL/6 Mouse Model Relevant for CVU Pathophysiology

C57/BL/6 mice were injected intraperitoneally seven times with 5 mg/2000 iron-dextran or 2000 PBS-Dextran (Sigma-Aldrich) on a three day interval. One day after the last iron injection, four 6 mm full-excisional wounds were inflicted with biopsy punchers (Stiefel) on the dorsal skin of shaved mice while under anesthesia. Wounds were photographed next to a lineal measure in order to quantify the wound areas using Adobe Photoshop software (Adobe Systems).

### IL-1β Quantitative PCR

Total RNA was isolated from human chronic venous leg ulcers (CVUs), murine wounds and corresponding healthy control skin using a commercial kit (RNeasy Microarray Tissue Mini Kit, Qiagen) as described by the manufacturer. Two μg of RNA per sample were reverse transcribed using illustra Ready-To-Go RT-PCR Beads (GE Healthcare). Quantity and quality of total RNA and cDNA were assessed using Nanodrop 1000 (Thermo Scientific) and QIAxcel Advance system (Qiagen). The 7300 real time PCR system (Applied Biosystems, Life Technologies) was used to amplify cDNA using Power SYBR green master mix (Applied Biosystems, Life Technologies). Primers specific for human IL-1β (FW: 5′-CCCAAGCAATACCCAAAGA-3′(SEQ ID NO: 6) and REV: 5′-CCACTTTGCTCTTGACTTCTA-3′(SEQ ID NO: 7)) and mouse IL-1β (FW: 5′-TCACAAGCAGAGCACAAG-3′(SEQ ID NO: 8) and REV: 5′-GAAACAGTCCAGCCCATAC-3′ (SEQ ID NO: 9)) were used for data given in Fig. S4.

### Effect of Human Recombinant IL-IRA Intradermal Injections on Delayed Wound Healing

Iron overload chronic wound healing model mice were randomly divided into three treatment groups including (i) Dextran/PBS acute wound healing control (ii) Iron/PBS group and (iii) Iron/rhIL-1RA treatment group with intradermal injections of 250 ng/wound recombinant human IL-1RA around the wound edges at days two and four as previously described for the acute model (36). Acute wound healing model mice were randomly assigned to (i) PBS-injected control group and (ii) rhIL-1RA treatment group as described for the chronic model. Wound closure over time was quantified by the wound surface area relative to day zero at days three, five, seven and ten (Fig. S5).

TABLE 1
Human skin donors. (A) Donor data of healthy skin used
in this study for in vivo characterization of ABCB5⁺
dermal cells and of CVU for IL-1β immunostaining
of CVU and normal human skin. (B) Donor data of healthy
skin used in this study for dermal cell ABCB5-sorting.

| | | Age at | | |
|---|---|---|---|---|
| Donor | Gender | biopsy | Skin biopsy | FIG. |
| ID | (m/f) | (yrs) | location | Nr(s). |
| A01 | M | 19 | Lower leg | 1B |
| A02 | F | 15 | Upper belly | 1B |
| A03 | F | 20 | Shoulder | 1B |
| A04 | F | 18 | Lower leg | 1B |
| A05 | M | 13 | High-parietal | 1B |
| A06 | f | 38 | Shoulder | 1B |
| A07 | f | 42 | Lumbal region | 1B |
| A08 | m | 33 | Lower back | 1B |
| A09 | m | 26 | Back | 1B |
| A10 | m | 38 | Neck | 1B |
| A11 | f | 74 | Shoulder | 1A |
| A12 | f | 16 | Gluteal region | 1A |
| A13 | f | 62 | Breast | 1C-D, |
| A14 | m | 73 | CVU + normal | S4C |
| | | | control (parallel | |
| | | | lower extremities) | |
| B01 | f | 58 | Behind left ear | 2A, 3B, 4B-E, |
| | | | | 5, 8B-C, S3 |
| B02 | f | 19 | Gluteal region | 2D-H (graphs) |
| B03 | m | 20 | Gluteal region | 2B-I (graphs + |
| | | | | pictures), S2 |
| B04 | f | 20 | Gluteal region | 2D-H (graphs) |
| B05 | f | 20 | Gluteal region | 2D-H (graphs) |
| B06 | f | 19 | Inside upper arm | 2D-H (graphs) |
| B07 | m | 27 | Upper arm | 2D-H (graphs) |
| B08 | f | 66 | Behind left ear | S3 |
| B09 | f | 51 | Behind left ear | S3 |
| B10 | m | 76 | Behind left ear | S3 |
| B11 | m | 51 | Behind left ear | S3 |
| B12 | f | 76 | Behind left ear | S3 |
| B13 | m | 51 | Behind left ear | not shown |
| B14 | m | 75 | Behind left ear | not shown |
| B01 + B08 + | — | — | Behind left ear | 6, 7, 8A, S3 |
| B09 + B10 + | | | | |
| B11 + B12* | | | | |

*Pooled-donor cell samples

TABLE 2
Release criteria for GMP-compliant dermal
ABCB5⁺ MSC preparations used in this study.

| Parameter | Test Method | Specification |
|---|---|---|
| Total count | Flow cytometry | Variable |
| viable cells | (2.7.29 E.P.) | |
| Cell vitality | Flow cytometry | ≥90% |
| Microbiological | (2.7.29 E.P.) | No growth |
| control | Adapted to 2.6.27 | |
| Endotoxin level | LAL-test | ≤2 EU/ml |
| Cell viability | (2.6.14 E.P.) | |
| CD90 surface | Flow cytometry | ≥75% |
| expression | Flow | |
| Bead residues | Flow cytometry | ≤0.5% |
| | Flow | |
| Content of | cytometry | ≥90% |
| ABCB5-positive | | |

TABLE 3
List of antibodies used in this study. A:
Primary antibodies used for immunostaining.

| | | | Applied | Company/ |
|---|---|---|---|---|
| Epitope | Clone | Species | reactivity | Reference |
| ABCB5 | 3C2-1D12 | Mouse IgG1 | Human, | (54) |
| (RFGAYLIQAGRMTPEG | | | mouse | |
| (SEQ ID NO: 1)) | | | | |
| Aggrecan | Polyclonal | Goat IgG | Human | R&D Systems |
| | | | | #AF1220 |
| CD31 CD68 CD206 | Polyclonal | Rabbit IgG | Human, | Abcam #28364 |
| | Y1/82A | Mouse IgG2b | mouse | BD #556059 |
| | Polyclonal | ΔRabbit | Human | Abcam #64693 |
| | | | Human | |
| F4/80 β2 | BM8 | Rat IgG2a | Mouse | eBioscience |
| microglobulin | Polyclonal | Δ Rabbit | Human | #14-4801-85 |
| | | | | [symbol] |
| IL-1β | Polyclonal | Rabbit | Human | Sdix |
| IL-1β | Polyclonal | Rabbit | Human, | Abcam #9722 |
| | | | mouse | |
| IL-1RA | EPR6483 | Rabbit IgG | Mouse | Abcam #124962 |
| NG2 | Polyclonal | Rabbit IgG | Human | Millipore |
| SOX2 | D6D9 | Rabbit IgG | Human | Cell Signaling |
| SSEA4 | Polyclonal | Rabbit IgG | Human | Bioss #bs- |
| TSG-6 | A38.1.20 | Rat IgG | Human | Santa Cruz sc- |
| TNFα | Polyclonal | Rabbit | Human | Abcam |
| | | | | #183896 |

[symbol]

indicates data missing or illegible when filed

B: Flow cytometry antibodies.

| | | | Applied | |
|---|---|---|---|---|
| Epitope | Clone | Species | reactivity | Company/Reference |
| (RFGAYLIQAGRMTPEG | 3C2-1D12 | Mouse | Human | (54) |
| (SEQ ID NO: 1)) | | IgG1 | | |
| CD14-PerCp | TÜK4 | Mouse | Human | Miltenyi Biotec |
| | | IgG2a | | #130-095-198 |
| CD20-PerCp | LT20.B4 | Mouse | Human | Miltenyi Biotec |
| | | IgG1 | | #130-095-198 |
| CD34-PerCp | AC136 | Mouse | Human | Miltenyi Biotec |
| | | IgG2a | | #130-095-198 |
| CD45-PerCp | 5B1 | Mouse | Human | Miltenyi Biotec |
| | | IgG2a | | #130-095-198 |
| CD68-FITC | eBioY1/82A | Mouse | Human | eBioscience |
| | | IgG2b Δ | | #11-0689-42 |
| CD73-APC | AD2 | Mouse | Human | Miltenyi Biotec |
| | | IgG1 | | #130-095-198 |
| CD90-FITC | DG3 | Mouse | Human | Miltenyi Biotec |
| | | IgG1 | | #130-095-198 |
| CD105-PE | 43A4E1 | Mouse | Human | Miltenyi Biotec |
| | | IgG1 | | #130-095-198 |
| CD133 | polyclonal | Rabbit IgG | Human | Abcam |
| | | | | #ab16518 |
| CD206- | 19.2 | Mouse | Human | eBioscience |
| eFluor450 | | IgG1 Δ | | #48-2069-41 |
| CD271-FITC | ME20.4-1.H4 | Mouse | Human | Miltenyi Biotec |
| | | IgG1 | | #130-91-917 |
| CD318 | polyclonal | Rabbit IgG | Human | Bioss |
| | | | | #5880-R |
| Dectin-1-PE | 15E2 | Mouse | Human | eBioscience |
| | | IgG2a Δ | | #12-9856-42 |
| IL-12/IL23 | EBioHP40 | Mouse | Human | eBioscience |
| p40-eFluor450 | | IgG1 Δ | | #48-7235-41 |
| MelanA | 1F12 | Mouse | Human | LifeSpan |
| | | IgG1 | | Biosciences |
| | | | | #LS-C174654 |
| SSEA4-PE | MC-813-70 | Mouse | Human | eBioscience |
| | | IgG3 | | #12-8843-71 |
| TNFα-PerCP-Cy5.5 | Mab11 | Mouse | Human | eBioscience |
| | | IgG1 Δ | | #45-7349-41 |
| Arginase 1-PE | polyclonal | Sheep IgG | Mouse | R&D Systems |
| | | | | #IC5868P |
| CD206-AF647 | C068C2 | Rat IgG2a | Mouse | BioLegend |
| | | Δ | | #141711 |
| Dectin-1-FITC | REA154 | Rec. | Mouse | Miltenyi Biotec |
| | | human | | #130-102-986 |
| | | IgG1 | | |
| IL12/IL23 | C17.8 | Rat IgG2a | Mouse | eBioscience |
| p40-PerCp-Cy5.5 | | Δ | | #45-7123-80 |
| NOS2-PE | CXNFT | Rat IgG2a | Mouse | eBioscience |
| | | Δ | | #12-5920-80 |
| TNF-α-PerCp-Cy5.5 | MP6-XT22 | Rat IgG1 | Mouse | BioLegend |
| | | Δ | | #506322 |

### REFERENCES

1 Lv F J, Tuan R S, Cheung K M et al. Concise review: the surface markers and identity of human mesenchymal stem cells. Stem Cells. 2014; 32:1408-1419.
2 Vishnubalaji R, Al-Nbaheen M, Kadalmani B et al. Skin-derived multipotent stromal cells—an archrival for mesenchymal stem cells. Cell and tissue research. 2012; 350:1-12.
3 Fukada S, Ma Y, Uezumi A. Adult stem cell and mesenchymal progenitor theories of aging. Frontiers in cell and developmental biology. 2014; 2:10.
4 Sen C K, Gordillo G M, Roy S et al. Human skin wounds: a major and snowballing threat to public health and the economy. Wound repair and regeneration: official publication of the Wound Healing Society [and] the European Tissue Repair Society. 2009; 17:763-771.
5 Schatton T, Yang J, Kleffel S et al. ABCB5 Identifies Immunoregulatory Dermal Cells. Cell reports. 2015.
6 Ksander B R, Kolovou P E, Wilson B J et al. ABCB5 is a limbal stem cell gene required for corneal development and repair. Nature. 2014; 511:353-357.
7 Sindrilaru A, Peters T, Wieschalka S et al. An unrestrained proinflammatory M1 macrophage population induced by iron impairs wound healing in humans and mice. The Journal of clinical investigation. 2011; 121:985-997.
8 Mirza R E, Fang M M, Ennis W J et al. Blocking interleukin-1beta induces a healing-associated wound macrophage phenotype and improves healing in type 2 diabetes. Diabetes. 2013; 62:2579-2587.
9 Sindrilaru A, Scharffetter-Kochanek K. Disclosure of the Culprits: Macrophages-Versatile Regulators of Wound Healing. Advances in wound care. 2013; 2:357-368.
10 Henderson J K, Draper J S, Baillie H S et al. Preimplantation human embryos and embryonic stem cells show comparable expression of stage-specific embryonic antigens. Stem Cells. 2002; 20:329-337.
11 Bartsch G, Yoo J J, De Coppi P et al. Propagation, expansion, and multilineage differentiation of human somatic stem cells from dermal progenitors. Stem cells and development. 2005; 14:337-348.
12 Battula V L, Bareiss P M, Treml S et al. Human placenta and bone marrow derived MSC cultured in serum-free, b-FGF-containing medium express cell surface frizzled-9 and SSEA-4 and give rise to multilineage differentiation. Differentiation; research in biological diversity. 2007; 75:279-291.
13 Gang E J, Bosnakovski D, Figueiredo C A et al. SSEA-4 identifies mesenchymal stem cells from bone marrow. Blood. 2007; 109:1743-1751.
14 Ozerdem U, Grako K A, Dahlin-Huppe K et al. NG2 proteoglycan is expressed exclusively by mural cells during vascular morphogenesis. Developmental dynamics: an official publication of the American Association of Anatomists. 2001; 222:218-227.
15 Dominici M, Le Blanc K, Mueller I et al. Minimal criteria for defining multipotent mesenchymal stromal cells. The International Society for Cellular Therapy position statement. Cytotherapy. 2006; 8:315-317.
16 Muraglia A, Cancedda R, Quarto R. Clonal mesenchymal progenitors from human bone marrow differentiate in vitro according to a hierarchical model. Journal of cell science. 2000; 113:1161-1166.
17 Vaculik C, Schuster C, Bauer W et al. Human dermis harbors distinct mesenchymal stromal cell subsets. The Journal of investigative dermatology. 2012; 132:563-574.
18 Pearson T, Greiner D L, Shultz L D. Creation of “humanized” mice to study human immunity. Current protocols in immunology/edited by John E Coligan [et al]. 2008; Chapter 15:Unit 15 21.
19 Phinney D G. Functional heterogeneity of mesenchymal stem cells: implications for cell therapy. J Cell Biochem. 2012; 113:2806-2812.
20 Driskell R R, Lichtenberger B M, Hoste E et al. Distinct fibroblast lineages determine dermal architecture in skin development and repair. Nature. 2013; 504:277-281.
21 Rinkevich Y, Walmsley G G, Hu M S et al. Identification and isolation of a dermal lineage with intrinsic fibrogenic potential. Science. 2015; 348.
22 Tsourdi E, Barthel A, Rietzsch H et al. Current Aspects in the Pathophysiology and Treatment of Chronic Wounds in Diabetes Mellitus. BioMed research international. 2013.
23 Mosser D M, Edwards J P. Exploring the full spectrum of macrophage activation. Nat Rev Immunol. 2008; 8:958-969.
24 Wietek C, O'Neill L A. Diversity and regulation in the NF-kappaB system. Trends in biochemical sciences. 2007; 32:311-319.
25 Ozes O N, Mayo L D, Gustin J A et al. NF-kappaB activation by tumour necrosis factor requires the Akt serine-threonine kinase. Nature. 1999; 401:82-85.
26 Cahill C M, Rogers J T. Interleukin (IL) 1beta induction of IL-6 is mediated by a novel phosphatidylinositol 3-kinase-dependent AKT/IkappaB kinase alpha pathway targeting activator protein-1. The Journal of biological chemistry. 2008; 283:25900-25912.
27 Nakamura K, Kawakami T, Yamamoto N et al. Activation of the NLRP3 inflammasome by cellular labile iron. Experimental hematology. 2016; 44:116-124.
28 Artlett C M. Inflammasomes in wound healing and fibrosis. J Pathol. 2013; 229:157-167.
29 Lee D J, Du F, Chen S W et al. Regulation and Function of the Caspase-1 in an Inflammatory Microenvironment. The Journal of investigative dermatology. 2015; 135:2012-2020.
30 He X, Mekasha S, Mavrogiorgos N et al. Inflammation and fibrosis during Chlamydia pneumoniae infection is regulated by IL-1 and the NLRP3/ASC inflammasome. J Immunol. 2010; 184:5743-5754.
31 Mirza R E, Fang M M, Weinheimer-Haus E M et al. Sustained inflammasome activity in macrophages impairs wound healing in type 2 diabetic humans and mice. Diabetes. 2014; 63:1103-1114.
32 Bitto A, Altavilla D, Pizzino G et al. Inhibition of inflammasome activation improves the impaired pattern of healing in genetically diabetic mice. British journal of pharmacology. 2014; 171:2300-2307.
33 Jiang D, Qi Y, Walker N G et al. The effect of adipose tissue derived MSCs delivered by a chemically defined carrier on full-thickness cutaneous wound healing. Biomaterials. 2013; 34:2501-2515.
34 Kim J, Hematti P. Mesenchymal Stem Cells Convert Human Macrophages to a Novel Type of Alternatively Activated Macrophages. Blood. 2009; 114:1403-1403.
35 Maggini J, Mirkin G, Bognanni I et al. Mouse bone marrow-derived mesenchymal stromal cells turn activated macrophages into a regulatory-like profile. Plos One. 2010; 5:e9252.
36 Qi Y, Jiang D, Sindrilaru A et al. TSG-6 released from intradermally injected mesenchymal stem cells accelerates wound healing and reduces tissue fibrosis in murine full-thickness skin wounds. The Journal of investigative dermatology. 2014; 134:526-537.
37 Zhang Q Z, Su W R, Shi S H et al. Human gingiva-derived mesenchymal stem cells elicit polarization of m2 macrophages and enhance cutaneous wound healing. Stem Cells. 2010; 28:1856-1868.
38 Nakajima H, Uchida K, Guerrero A R et al. Transplantation of Mesenchymal Stem Cells Promotes an Alternative Pathway of Macrophage Activation and Functional Recovery after Spinal Cord Injury. J Neurotraum. 2012; 29:1614-1625.
39 Ishida Y, Kondo T, Kimura A et al. Absence of IL-1 receptor antagonist impaired wound healing along with aberrant NF-kappaB activation and a reciprocal suppression of TGF-beta signal pathway. J Immunol. 2006; 176:5598-5606.
40 Thomay A A, Daley J M, Sabo E et al. Disruption of Interleukin-1 Signaling Improves the Quality of Wound Healing. Am J Pathol. 2009; 174:2129-2136.
41 Meier R P, Mahou R, Morel P et al. Microencapsulated human mesenchymal stem cells decrease liver fibrosis in mice. J Hepatol. 2015; 62:634-641.
42 Dripps D J, Brandhuber B J, Thompson R C et al. Interleukin-1 (IL-1) receptor antagonist binds to the 80-kDa IL-1 receptor but does not initiate IL-1 signal transduction. The Journal of biological chemistry. 1991; 266:10331-10336.
43 Liu S, Jiang L, Li H et al. Mesenchymal stem cells prevent hypertrophic scar formation via inflammatory regulation when undergoing apoptosis. J Invest Dermatol. 2014; 134:2648-2657.
44 Nemeth K, Leelahavanichkul A, Yuen P S et al. Bone marrow stromal cells attenuate sepsis via prostaglandin E(2)-dependent reprogramming of host macrophages to increase their interleukin-10 production. Nature medicine. 2009; 15:42-49.
45 Luu N T, McGettrick H M, Buckley C D et al. Crosstalk between mesenchymal stem cells and endothelial cells leads to downregulation of cytokine-induced leukocyte recruitment. Stem Cells. 2013; 31:2690-2702.
46 Peters T, Sindrilaru A, Hinz B et al. Wound-healing defect of CD18(−/−) mice due to a decrease in TGF-beta1 and myofibroblast differentiation. The EMBO journal. 2005; 24:3400-3410.
47 Frank N Y, Pendse S S, Lapchak P H et al. Regulation of progenitor cell fusion by ABCB5 P-glycoprotein, a novel human ATP-binding cassette transporter. The Journal of biological chemistry. 2003; 278:47156-47165.
48 Singh K, Krug L, Basu A et al. Alpha-Ketoglutarate Curbs Differentiation and Induces Cell Death in Mesenchymal Stromal Precursors with Mitochondrial Dysfunction. Stem Cells. 2017; 35:1704-1718.
49 Gregory C A, Gunn W G, Peister A et al. An Alizarin red-based assay of mineralization by adherent cells in culture: comparison with cetylpyridinium chloride extraction. Analytical biochemistry. 2004; 329:77-84.
50 Singh K, Maity P, Krug L et al. Superoxide anion radicals induce IGF-1 resistance through concomitant activation of PTP1B and PTEN. EMBO molecular medicine. 2015; 7:59-77.
51 Singh K, Camera E, Krug L et al. JunB defines functional and structural integrity of the epidermo-pilosebaceous unit in the skin. Nature communications. 2018; 9:3425.

All references cited herein are fully incorporated by reference. Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. A composition, comprising:
a population of synthetic ABCB5+ stem cells, wherein greater than 96% of the population is an in vitro progeny of physiologically occurring skin-derived ABCB5-positive mesenchymal stem cells.

2. The composition of claim 1, wherein greater than 96.5%, 97%, 97.5%, 98%, 98.5%, 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells.

3. The composition of claim 1, wherein 100% of the population is an in vitro progeny of physiologically occurring skin-derived ABCB5-positive mesenchymal stem cells.

4. The composition of claim 1, wherein greater than 90% of the synthetic stem cells in the population co-express CD90.

5. The composition of claim 1, wherein the population of synthetic stem cells are capable of VEGF secretion under hypoxia as measured by ELISA.

6. The composition of claim 1, wherein the population of synthetic stem cells are capable of IL-1RA secretion after co-culture with Mi-polarized macrophages.

7. The composition of claim 1, wherein the population of synthetic stem cells induce decreased TNF-alpha and IL-12/IL-23p40 secretion, and increased IL-10 secretion, in macrophage co-culture relative to isolated physiologically occurring skin-derived ABCB5-positive mesenchymal stem cells.

8. The composition of claim 1, wherein the population of synthetic stem cells possess multipotent differentiation capacity.

9. The composition of claim 1, wherein the population of synthetic stem cells possess the capacity to differentiate into cells derived from all three germ layers, endoderm, mesoderm and ectoderm.

10. The composition of claim 1, wherein the population of synthetic stem cells possess corneal epithelial differentiation capacity.

11. The composition of claim 1, wherein the population of synthetic stem cells exhibit increased expression of stem cell markers including SOX2, NANOG and SOX3 relative to isolated physiologically occurring skin-derived ABCB5-positive mesenchymal stem cells.

12. The composition of claim 1, wherein the population of synthetic stem cells exhibit decreased expression of mesenchymal stromal differentiation markers including MCAM, CRIG1 and ATXN1 relative to isolated physiologically occurring skin-derived ABCB5-positive mesenchymal stem cells.

13. The composition of claim 1, wherein at least 5% of the population of synthetic stem cells includes an exogenous gene.

14-19. (canceled)

20. A method for preparing a population of cells, comprising: isolating a primary cells from skin tissue from a human subject; culturing the primary cells in culture medium until the cells produce enough progeny to reach greater than 60% confluence of mixed cells, harvesting the mixed cells, culturing the harvested mixed cells, reharvesting and culturing the cells through at least 5 passages until the population of cells reaches at least 99% manufactured synthetic cells and less than 10% is primary physiologically occurring skin-derived cells; and isolation of ABCB5-positive cells using an ABCB5+ antibody.

21. The method of claim 20, wherein the method involves reharvesting and culturing the cells through at least 6, 7, 8, 9, 10, 11, 12, 13, 14, 15, or 16 passages.

22. The method of claim 20, wherein the method involves reharvesting and culturing the cells until the population of cells reaches at least 99.99% manufactured synthetic cells and less than 0.01% is primary physiologically occurring skin-derived cells.

23. The method of claim 20, wherein the method involves reharvesting and culturing the cells until the population of cells reaches at least 99.9995% manufactured synthetic cells and less than 0.0005% is primary physiologically occurring skin-derived cells.

24. The method of claim 20, wherein the method involves reharvesting and culturing the cells until the population of cells reaches at least 99.999997% manufactured synthetic cells and less than 0.000003% is primary physiologically occurring skin-derived cells.

25-29. (canceled)

30. A method for inducing tissue generation, comprising promoting differentiation of an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells into a differentiated tissue.

31. A method for promoting syngeneic transplants comprising administering to a subject having a syngeneic transplant an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells.

32. A method for treating peripheral arterial occlusive disease (PAOD), comprising administering to a subject having PAOD an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells in an effective amount to treat the disease.

33. A method for treating acute-on-chronic liver failure (AOCLF), comprising administering to a subject having AOCLF an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells in an effective amount to treat the disease.

34. A method for treating limbal stem cell deficiency (LSCD), comprising administering to a subject having LSCD an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells in an effective amount to treat the disease.

35-54. (canceled)

55. A method of treating a hyper-inflammatory disorder, comprising administering to a subject having a hyper-inflammatory disorder, an effective amount of an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells to treat the hyper-inflammatory disorder.

56. The method of claim 55, wherein the hyper-inflammatory disorder is a disorder associated with a virally induced cytokine storm.

57. The method of claim 56, wherein the subject has a SARS infection.

58. The method of claim 55, wherein the hyper-inflammatory disorder is a disorder associated with sepsis, systemic inflammatory response syndrome (SIRS), cachexia, septic shock syndrome, traumatic brain injury (e.g., cerebral cytokine storm), graft versus host disease (GVHD), or the result of treatment with activated immune cells, e.g., IL-2 activated T cells, T cells activated with anti-CD19 Chimeric Antigen Receptor (CAR) T cells.

59. A method of treating a SARS infection in a subject, comprising administering to the subject, an effective amount of an isolated population of synthetic ABCB5+ stem cells to treat the SARS infection in the subject.

60. The method of claim 59, wherein the SARS infection is a SARS-CoV-2 infection.

61. The method of claim 59, wherein greater than 99%, 99.5%, 99.7%, 99.9%, 99.99%, 99.998%, 99.999%, or 99.999997% of the population of synthetic ABCB5+ stem cells is an in vitro progeny of physiologically occurring skin-derived ABCB5-positive mesenchymal stem cells.

62. (canceled)

63. A method of treating a subject having an infectious disease, comprising identifying a subject having an infectious disease and at risk of or having a cytokine storm associated with an infectious disease; and administering an isolated population of synthetic ABCB5+ stem cells to treat the subject.

64. The method of claim 63, wherein the infectious disease is caused by a coronavirus.

65. The method of claim 63, wherein the coronavirus is SARS-CoV-2.

66. The method of claim 63, wherein greater than 99%, 99.5%, 99.7%, 99.9%, 99.99%, 99.998%, 99.999%, or 99.999997% of the population of synthetic ABCB5+ stem cells is an in vitro progeny of physiologically occurring skin-derived ABCB5-positive mesenchymal stem cells.

67. (canceled)
